SEQUENCE LISTING (20 sequences)

SEQ ID NO: 1 - protein, 190 aa, Artificial Sequence
  Synthetic Construct; Canine OX40 polypeptide
MEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTKLYLMKRRTMQPASKSSDAVCEDRSLPATLPWETQSPLTRPPTPQPTMAWPRTSQGPFTPPTEPPRGPQGS

SEQ ID NO: 2 - protein, 159 aa, Artificial Sequence
  Synthetic Construct; VH domain - OX40 antibody
DVLLVESGGDLVKPGGTLRLSCVASGFPFSNFNMGWVRQAPGKGLQWVAWIHGSGMTTRYADDVTGRFTISRDNAKDTLYLEMDSLRLEDTAKYYCARDLDDAYLGPNWFSYWGQGTLVIVSSASTTAPSVFPLAPSCGSTSGSTVALACLVSGYFPEP

SEQ ID NO: 3 - protein, 112 aa, Artificial Sequence
  Synthetic Construct; VL domain - OX40 antibody
DIVMTQAPPSLSLSPGEPASISCKASQSLLHSNGNTYLYWFRQKPGQSPEGLIYKVSDRFTGVSDRFSGSGSGTDFTLRISRVEADDAGVYYCGQNLQLPYSFSQGTKLEIK

SEQ ID NO: 4 - protein, 10 aa, Artificial Sequence
  Synthetic Construct; CDRH1 region - OX40 antibody
GFPFSNFNMG

SEQ ID NO: 5 - protein, 16 aa, Artificial Sequence
  Synthetic Construct; CDRH2 region - OX40 antibody
IHGSGMTTRYADDVTG

SEQ ID NO: 6 - protein, 14 aa, Artificial Sequence
  Synthetic Construct; CDRH3 region - OX40 antibody
DLDDAYLGPNWFSY

SEQ ID NO: 7 - protein, 16 aa, Artificial Sequence
  Synthetic Construct; CDRL1 region - OX40 antibody
KASQSLLHSNGNTYLY

SEQ ID NO: 8 - protein, 7 aa, Artificial Sequence
  Synthetic Construct; CDRL2 region - OX40 antibody
KVSDRFT

SEQ ID NO: 9 - protein, 9 aa, Artificial Sequence
  Synthetic Construct; CDRL3 region - OX40 antibody
GQNLQLPYS

SEQ ID NO: 10 - protein, 299 aa, Artificial Sequence
  Synthetic Construct; Heavy chain constant region (CH) - OX40 antibody
VTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 11 - protein, 110 aa, Artificial Sequence
  Synthetic Construct; Light chain constant region (CL) - OX40 antibody
RTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 12 - protein, 21 aa, Artificial Sequence
  Synthetic Construct; Leader peptide
MRAWIFFLLCLAGRALAAPLA

SEQ ID NO: 13 - DNA, 599 bp, Artificial Sequence
  Synthetic Construct; OX40-6His optimized cDNA
ccatggaacataactgctttggcaatacctatccgaaagatggcaaatgttgcaatgattgcccgccgggttatggtatggaaagccgttgcagtcgtagtcatgataccaaatgtcatcagtgtccgagtggtttttataatgaagccaccaattatgaaccgtgtaaaccgtgcacccagtgtaatcagcgcagtggcagtgaaccgaaacgccgctgcaccccgacccaggataccatttgcagctgcaaaccgggcaccgaaccgcgtgatggttataaacgcggtgttgattgtgccccgtgcccgccgggccattttagtccgggtgatgatcaggcctgtaaaccgtggaccaaactgtatctgatgaaacgtcgcaccatgcagccggccagcaaaagtagcgatgcagtttgcgaagatcgtagcctgccggcaaccctgccgtgggaaacccagagcccgctgacccgtccgccgacacctcagcctacaatggcttggccgcgcaccagccagggcccttttaccccgccgaccgaaccgccgcgcggtcctcagggtagtcatcatcatcatcaccattaactcgag

SEQ ID NO: 14 - DNA, 1440 bp, Artificial Sequence
  Synthetic Construct; VH-CH123
atgagggcctggatcttctttctcctttgcctggccgggagggctctggcagccccgctagcagatgtgctgctcgtggaaagcggcggcgatctggtgaagcccggtggcacactgaggctgagctgtgtggccagcggcttccccttcagcaacttcaacatgggctgggtgaggcaagctcccggtaaaggtttacagtgggtggcttggattcatggcagcggcatgaccactcgttacgccgacgacgtgaccggtcgtttcaccatctctcgtgacaacgccaaggacactttatatttagagatggactctttaaggctggaggacaccgccaagtactactgcgccagagatttagacgacgcctatttaggccccaactggttcagctactggggccaaggtactttagtgatcgtgagcagcgctagcaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccagtgcccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaggcccatcaacccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaatga

SEQ ID NO: 15 - DNA, 732 bp, Artificial Sequence
  Synthetic Construct; VL-CK
atgagggcctggatcttctttctcctttgcctggccgggagggctctggcagccccgctagcagacatcgtgatgacccaagctcctccttctttatctttaagccccggtgagcccgctagcatcagctgcaaggccagccagtctttactgcacagcaacggcaacacctatttatactggtttcgtcagaagcccggacagagccccgaaggtttaatctacaaggtgagcgatcgtttcaccggcgtgagcgacagatttagcggcagcggtagcggcaccgatttcactttaaggatctctcgtgtggaggccgatgatgccggcgtgtactactgcggccagaatttacagctgccctacagcttcagccaaggtaccaagctggagatcaagcgtacggatgcccagccagccgtctatttgttccaaccatctccagaccagttacacacaggaagtgcctctgttgtgtgtttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggactaa

SEQ ID NO: 16 - protein, 479 aa, Artificial Sequence
  Synthetic Construct; VH1-CH123 (Leader-VH- Ch123, IgGb Type)
MRAWIFFLLCLAGRALAAPLADVLLVESGGDLVKPGGTLRLSCVASGFPFSNFNMGWVRQAPGKGLQWVAWIHGSGMTTRYADDVTGRFTISRDNAKDTLYLEMDSLRLEDTAKYYCARDLDDAYLGPNWFSYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 17 - protein, 243 aa, Artificial Sequence
  Synthetic Construct; VK2-CK (Leader-VL-CK)
MRAWIFFLLCLAGRALAAPLADIVMTQAPPSLSLSPGEPASISCKASQSLLHSNGNTYLYWFRQKPGQSPEGLIYKVSDRFTGVSDRFSGSGSGTDFTLRISRVEADDAGVYYCGQNLQLPYSFSQGTKLEIKRTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 196 aa, Artificial Sequence
  Synthetic Construct; OX40-6His polypeptide
MEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTKLYLMKRRTMQPASKSSDAVCEDRSLPATLPWETQSPLTRPPTPQPTMAWPRTSQGPFTPPTEPPRGPQGSHHHHHH

SEQ ID NO: 19 - protein, 272 aa, Artificial Sequence
  Synthetic Construct; canine OX40 polypeptide
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTKLYLMKRRTMQPASKSSDAVCEDRSLPATLPWETQSPLTRPPTPQPTMAWPRTSQGPFTPPTEPPRGPQLAAVLGLGLGLLAPVAAALALLLHHRAWRLPPGGNSFRTPIQEEHADANSTLAKIGS

SEQ ID NO: 20 - protein, 278 aa, Artificial Sequence
  Synthetic Construct; OX40-6His Polypeptide
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTKLYLMKRRTMQPASKSSDAVCEDRSLPATLPWETQSPLTRPPTPQPTMAWPRTSQGPFTPPTEPPRGPQLAAVLGLGLGLLAPVAAALALLLHHRAWRLPPGGNSFRTPIQEEHADANSTLAKIGSHHHHHH

(19) United States
(12) Patent Application Publication
(10) Pub. No.: US 2022/0332837 A1
(43) Pub. Date: Oct. 20, 2022

(54) METHODS AND COMPOSITIONS FOR OX40 ACTIVATION IN TREATMENT OF CANINE CANCER

(71) Applicant: COLORADO STATE UNIVERSITY RESEARCH FOUNDATION, Fort Collins, CO (US)

(72) Inventors: Steven Dow, Fort Collins, CO (US); Leone Sophia Hopkins, Fort Collins, CO (US); Dylan Ammons, Fort Collins, CO (US); Lyndah Chow, Fort Collins, CO (US)

(21) Appl. No.: 17/716,507

(22) Filed: Apr. 8, 2022

Related U.S. Application Data
(60) Provisional application No. 63/172,569, filed on Apr. 8, 2021.

(51) Int. Cl. C07K 16/28; A61K 39/395; A61K 45/06; A61P 35/00

(52) U.S. Cl. CPC C07K 16/2878; A61K 39/3955; A61K 45/06; A61P 35/00; C07K 2317/75; C07K 2317/622

(57) ABSTRACT

Embodiments of the present invention relate to compositions and methods for activating canine OX40 to treat a condition in dogs. In certain embodiments, the condition can be cancer or an immunosuppressed condition in dogs and OX40 activation treats, ameliorates or prevents onset or progression of the condition. In other embodiments, compositions disclosed herein generally relate to compositions including, but not limited to, activating antibodies having specific affinity for OX40 for inducing activity of canine OX40. In other embodiments, compositions disclosed herein can be used for treating certain cancers or an immunosuppressed condition to treat the cancer or alleviate immunosuppression in dogs. In certain embodiments, other immune activators can be administered in combination with OX40 activating antibodies such as Toll-Like Receptor (TLR) ligands alone or in combination with other non-specific immunostimulant agents.

### PRIORITY

This US Non-Provisional Application claims the benefit of priority pursuant to 35 U.S.C. § 119(e) to U.S. Provisional Patent Application No. 63/172,569 filed on Apr. 8, 2021. This provisional application is hereby incorporated by reference in its entirety for all purposes.

### FIELD

Embodiments of the present invention generally relate to compositions and methods for activating canine OX40 to treat a condition in dogs. In certain embodiments, the condition can be cancer or an immunosuppressed condition in dogs and OX40 activation treats, ameliorates or prevents onset or progression of the condition. In other embodiments, compositions disclosed herein can be used for treating certain cancers or an immunosuppressed condition in order to treat the cancer or alleviate immunosuppression in dogs.

### SEQUENCE LISTING

This US Non-Provisional Application contains a sequence listing which has been submitted electronically in ASCII format and is hereby incorporated by reference in its entirety herein for all purposes. The ASCII copy, created on Apr. 7, 2022, is named 065620-721643_CSURF18-013_Sequence Listing_ST25.txt and is 31 kilobytes in size.

### BACKGROUND

Cancer and immunosuppressive conditions in the canine population continue to be a major concern. While some specific therapies have been developed, the distinct physiology and unique characteristics of the canine population as well as the growing need for canine cancer treatments have made more generally applicable treatments desirable. Accordingly, there is a need for other methods of treating cancer that are generally applicable to all tumors including solid tumors, as well as more generally applicable treatments for immunosuppressive conditions, in dogs.

### SUMMARY

Embodiments of the present disclosure are based, at least in part, on the development of isolated activating anti-canine OX40 activating antibodies and their uses to treat conditions in dogs. In accordance with these embodiments. these activating OX40 antibodies have a high binding affinity and specificity to canine OX40 and can activate OX40 to increase OX40 activity by inducing immunity and/or treating cancer in canines.

In some embodiments, an isolated activating anti-canine OX40 antibody provided herein can include a V_{H} having about 80-85%, about 90% or more, about 95% or more, about 99% or up to 100% sequence identity to a V_{H} of monoclonal antibody clone 7E10F3 or monoclonal antibody clone CAF7 and a V_{L} having about 80-85%, about 90% or more, about 95% or more, about 99% or up to 100% sequence identity to a V_{L} of monoclonal antibody clone 7E10F3 or monoclonal antibody clone CAF7. In other embodiments, the isolated activating antibody can be a monoclonal antibody clone 7E10F3 or monoclonal antibody clone CAF7.

In other embodiments, an isolated activating anti-canine OX40 antibody disclosed herein binds to one or more epitope of a polypeptide having an amino acid sequence represented by SEQ ID NOs: 1 and 19, where binding the one or more epitope activates at least one OX40 function(s). In some embodiments, an isolated activating anti-canine OX40 antibody does not inhibit OX40 activity. In yet other embodiments, an isolated activating anti-canine antibody is a full-length antibody or an antigen binding fragment thereof. In some embodiments, an isolated activating anti-canine antibody is a monoclonal antibody. In certain embodiments, the isolated activating anti-canine antibody is a single-chain antibody (scFv).

In other embodiments, a polynucleotide encoding an activating anti-canine OX40 antibody or antigen binding fragment thereof provided herein is contemplated. In accordance with these embodiments, the polynucleotide can further include a vector for expressing the encoded antibody or antigen binding fragment. In yet other embodiments, host cells having the polynucleotide encoding an activating anti-canine OX40 antibody or antigen binding fragment thereof are included herein.

In other embodiments, methods of identifying an activating antibody of OX40, are disclosed including, but not limited to: (i) culturing a host cell provided herein under conditions allowing for expression of an antibody that binds OX40, or fragment thereof; (ii) harvesting the antibody produced from the cell culture; and (iii) screening for activity of OX40. In certain embodiments, screening for activity of OX40 includes, but is not limited to. detecting an increase in non-regulatory T cell activity, proliferation and/or survival; or detecting a decrease in regulatory T cell activity, proliferation and/or survival in a canine after administration of the anti-canine OX40 antibody to the canine provided herein.

In some embodiments, pharmaceutical compositions are disclosed containing at least one activating anti-canine OX40 antibody or a polynucleotide expressing the at least one activating anti-canine OX40 antibody as described herein and a pharmaceutically acceptable carrier of use for treating conditions in canines. In accordance with these embodiments, the pharmaceutical compositions can further include a non-specific innate immune response stimulator, which can include one or more cationic liposome, one or more TLR ligand, and one or more cellular adhesion agent. In some embodiments, the pharmaceutical compositions can further include one or more of an anti-microbial agent, a chemotherapeutic agent, and/or an anti-PD-1 antibody. In yet other embodiments, the composition or pharmaceutical composition disclosed herein does not include an inhibitor of OX40 activity.

In other embodiments, methods for using pharmaceutical compositions including, but not limited to, activating anti-canine OX40 antibody for treating a condition in dogs is contemplated. In some embodiments, methods include inducing innate immunity in a canine to boost immune response or treat an immunosuppressive condition. In accordance with these embodiments, pharmaceutical compositions described herein can be administered to the canine by any method known in the art. In another embodiment, methods are provided for treating cancer in a canine. In accordance with these embodiments, methods for treating cancer in a canine include, but are not limited to, administering to the canine in need thereof a therapeutically effective amount of the pharmaceutical composition described herein. In other embodiments, cancer includes, but is not limited to, a solid tumor. In certain embodiments, when treating solid tumors, the pharmaceutical compositions can be administered systemically, topically or by direct local administration into the solid tumor of the canine.

In other embodiments, methods disclosed herein concern treating immunosuppression in a canine. In accordance with these embodiments, the canine can be administered a pharmaceutical composition containing activating anti-canine OX40 antibody disclosed herein alone or in combination with other treatments or other agents. In some embodiments, immunosuppression of the canine can be caused by a microbial or mixed microbial infection such as a viral, a fungal, a bacterial, or a protozoal infection or a combination thereof. In other embodiments, the microbial infection can include be a chronic infection. In other embodiments, the microbial infection can be an acute infection.

In other embodiments, therapeutic methods provided herein can further include administering a secondary, alternative, or standard treatment to the canine of at least one of before, during, or after administering the pharmaceutical compositions disclosed herein. In certain embodiments, the secondary or standard or alternative can include, but is not limited to, radiation therapy, a TLR agonist administration alone or in combination with other agents, an anti-PD1 antibody, a chemotherapeutic agent, an antimicrobial such as an antibiotic, an antiviral, or an antifungal, or a combination of any thereof.

### BRIEF DESCRIPTION OF THE DRAWINGS

FIG. 1 illustrates an example of antitumor activity of an OX40 antibody versus a control antibody in an animal model implanted with tumor cells in accordance with certain aspects of the present disclosure.

FIGS. 2A and 2B illustrate exemplary plots of flow cytometry data of naïve, untreated (2A) OX-40 expressing peripheral blood mononuclear cells (PBMCs) or OX-40 (2B) expressing PBMCs in accordance with certain aspects of the present disclosure.

FIGS. 3A and 3B illustrates an example of flow cytometry detected expression of OX-40 in a mammalian cell line where control cells are illustrated (3A) and OX-40 mAb-treated cells (3B) in accordance with certain aspects of the present disclosure.

FIG. 4 illustrates an example of a time course of OX-40 expression in canine T cells activated with a T-cell stimulus in accordance with certain aspects of the present disclosure.

FIGS. 5A and 5B illustrate an example of flow cytometry plots indicating levels of CD25 (5A) and intracellular IFNγ (5B) detected in a canine CD5+ T cell population after stimulation with a control antibody (first column), an OX-40 antibody (middle column), or a positive control in accordance with certain aspects of the present disclosure.

FIGS. 6A and 6B illustrate an example of immunohistocytochemistry images of tumor sections isolated from a canine cancer biopsy and immunostained with an OX40 antibody (6A) or with control antibody (6B) in accordance with certain aspects of the present disclosure.

FIG. 7 illustrates a clinical trial design that assesses the impact of OX40 antibody as an anti-tumor treatment in dogs in accordance with certain aspects of the present disclosure.

FIGS. 8A and 8B are a graph (A) and images (B) indicating levels of intratumoral regulatory T cells (Tregs) in dogs treated with an OX40 antibody relative to a control treatment and an example of immunohistocytochemistry images of tumor sections isolated from the treated dogs in accordance with certain aspects of the present disclosure.

FIG. 9 illustrates an example of levels depicted in a graph of certain expression of genes in tumor cells isolated from a mammal treated with an OX40 antibody or a control treatment in accordance with certain aspects of the present disclosure.

FIG. 10 illustrates a canine OX40 cDNA sequence (SEQ ID NO: 13) and the expected protein product (SEQ ID NO: 18) in accordance with certain aspects of the present disclosure.

FIG. 11 illustrates a schematic of a pcDNA expression vector expressing a recombinant canine OX40 in E. coli in accordance with certain aspects of the present disclosure.

FIG. 12 illustrates an example of a blot illustrating expression of recombinant OX40 by gel separation according to size in accordance with certain aspects of the present disclosure.

FIG. 13 illustrates an example of an immunoblot demonstrating an OX40 antibody present and visualized by gel separation according to size in accordance with certain aspects of the present disclosure.

FIG. 14 illustrates amino acid sequences of full heavy and light chains of illustrative canine OX40 antibodies depicting certain regions and amino acid sequence comparisons in accordance with certain aspects of the present disclosure.

FIG. 15 illustrates annotated amino acid sequences of the full heavy (SEQ ID NO: 16) and light (SEQ ID NO: 17) chains of an illustrative OX40 antibody described herein where the leader sequence is in green, the variable sequence is in grey, and the constant sequence is in cyan (colored images available upon request) in accordance with certain aspects of the present disclosure.

FIG. 16 illustrates an example of an immunoblot of a gel separation illustrating an OX40 antibody run after non-reducing and after reducing conditions in accordance with certain aspects of the present disclosure.

FIG. 17 illustrates an exemplary treatment regimen for combination therapies of radiotherapy and OX40 antibody therapy in accordance with aspects of the present disclosure.

FIGS. 18A-18B are representative three-dimensional OX40 injection map images in experimental canines in accordance with certain aspects of the present disclosure.

FIG. 19 illustrates representative immunohistochemistry (IHC) images of immune cell infiltrates related to certain treatments disclosed regarding radiotherapy and immunotherapy-treated tumors in accordance with certain aspects of the present disclosure.

FIG. 20 illustrates data demonstrating fold-change in percent positive immune cell density following radiotherapy alone or in combination with immunotherapy treatments in accordance with certain aspects of the present disclosure.

FIGS. 21A-21D illustrates data regarding changes in regulatory T cell gene expression following radiotherapy only or in combination with immunotherapy treatments (A); B illustrates data regarding changes in exhausted T cell gene expression following radiotherapy and immunotherapy tumor treatments; C illustrates data regarding changes in myeloid cell gene expression following radiotherapy and immunotherapy tumor treatments; and D illustrates data regarding changes in effector T cell gene expression following radiotherapy and immunotherapy treatments in accordance with certain aspects of the present disclosure.

FIG. 22 illustrates data regarding fold-changes in serum cytokine levels of certain cytokines following radiotherapy alone or in combination with immunotherapy tumor treatments in accordance with certain aspects of the present disclosure.

FIGS. 23A-23B illustrate data regarding fold-changes in density of CD31+ endothelial cells following radiotherapy alone or in combination with immunotherapy treatments (23A); and 23B illustrates data regarding fold-changes in tumor hemoglobin saturation following radiotherapy alone or in combination with immunotherapy treatments in accordance with certain aspects of the present disclosure. Definitions

As used herein, the term “about,” can mean relative to the recited value plus and/or minus, e.g., amount, dose, temperature, time, percentage, etc., ±10%, ±9%, ±8%, ±7%, ±6%, ±5%, ±4%, ±3%, ±2%, or ±1%.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

An “epitope” refers to the site on a target antigen that is recognized and bound by an antibody. In some embodiments, the site can be entirely composed of amino acid components, entirely composed of chemical modifications of amino acids of the protein (e.g., glycosyl moieties), or composed of combinations thereof. In some embodiments, overlapping epitopes can include at least one common amino acid residue. In some embodiments, an epitope herein can be linear, (e.g., about 5-50 amino acids in length). In some embodiments, an epitope herein can be conformational. The epitope to which an antibody binds can be determined by routine technology, for example, the epitope mapping method (see, e.g., descriptions below). In some embodiments, two or more OX40 antibodies described herein can bind to the same epitope or a substantially overlapping epitope (e.g., containing less than 3 non-overlapping amino acid residues, less than 2 non-overlapping amino acid residues, or only 1 non-overlapping amino acid residue). Whether two antibodies compete against each other from binding to the cognate antigen can be determined by a competition assay, which is well known in the art.

Certain constructs and polypeptides are described below in terms of “percent identity” or “percent sequence identity” to a reference sequence. When used herein, the term “percent identity” or “percent sequence identity” of two amino acid sequences can be determined using an algorithm of any methods known in the art.

Constructs and polypeptides having a certain percent sequence identity to a reference sequence can have one or more “conservative amino acid substitutions” relative to the reference sequence. These “conservative amino acid substitution(s)” can refer to one or more amino acid substitution that does not alter the relative charge or size characteristics of the protein in which the amino acid substitution is made. In some embodiments, variants herein can be prepared according to methods for altering polypeptide sequence known to one of ordinary skill in the art.

### DETAILED DESCRIPTION

In the following sections, certain exemplary compositions and methods are described to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Embodiments of the instant disclosure relate to novel isolated activating antibodies for inducing activation of OX40 alone or in combination therapies to treat canine tumors or cancer or other immunosuppressive conditions. OX40 belongs to the tumor necrosis factor receptor superfamily that is expressed primarily on CD4+ T cells and regulatory T cells and can act as a co-stimulatory checkpoint molecule. Through interaction with its ligand OX40L, OX40 can exert anti-tumor immune effects. Under certain conditions, OX40 inhibits T cell (Tregs) generation and function and affects removal of the Treg immune suppressive effects within tumor microenvironments.

In some embodiments, radiation therapy (e.g., SBRT) in combination with OX40 antibody administration to canines are contemplated herein for improved treatment regimens. In certain embodiments, an OX40 activating antibody can be administered at the same time as radiotherapy. In accordance with these embodiments, OX40 activating antibody can be administered within a timeframe during which one or more radiotherapy sessions can be performed. In some embodiments, the OX40 activating antibody can be administered after radiotherapy treatment. In other embodiments, the OX40 activating antibody can be administered before radiotherapy treatment. In other embodiments, radiotherapy treatment can include about 2-20 grays (Gy) per treatment. In certain embodiments, radiotherapy treatment can include about 6-10 Gy per treatment. In yet other embodiments, the radiotherapy can include 1 to 7 or 1 to 3 or 2 to treatments. In certain embodiments, the radiotherapy can be 3 to 5 sessions.

In some embodiments, the isolated activating anti-canine OX40 antibodies disclosed herein are capable of binding to OX40 expressed on a canine cell surface to induce one or more OX40 activities. In accordance with these embodiments, activating OX40 can have beneficial effects including, but not limited to, enhancing T cell function (e.g., cytokine secretion), T cell survival and T cell proliferation. In other embodiments, OX40 activation can trigger reduction to full depletion of regulatory T cells which can reduce effects that block anti-tumor immune responses to enhance immune responses against tumors. In yet other embodiments, activated OX40 activities can include a combination of these effects. In accordance with these embodiments, the activating antibodies disclosed herein can be used to enhance anti-tumor responses in canines to shrink or eliminate a cancerous growth and/or reduce or prevent tumor expansion or tumor cell migration. In certain embodiments, the activating antibodies disclosed herein can be used in conjunction with another active agent or treatment (e.g., a chemotherapeutic agent, an antibiotic, an anti-fungal, an anti-viral, or a non-specific immune stimulator, radiation therapy). In some embodiments, compositions disclosed herein can be used to reduce or eliminate the need for more toxic agents or treatment such as radiation or chemotherapy.

In some embodiments, OX40 activating antibodies can be administered in combination with a non-specific immune stimulator composition, containing, for example, a TLR3, TLR4, and/or TLR9 ligand. In accordance with these embodiments, these combinations of the activating antibodies and a second active agent can act synergistically to enhance immunity in a dog and/or treat cancer. In other embodiments, polynucleotides, and expression systems for producing the antibodies described herein as well as kits for their use are provided.

In certain embodiments, methods for treating cancers (e.g., solid tumor cancers) and additional immune system enhancers (e.g., a non-specific immune stimulating composition) can be used in combination, simultaneously or sequentially with one after the other or other regimen. In some embodiments, a non-specific immune stimulating composition containing one or more cationic liposome, one or more TLR ligand (e.g., TLR3, TLR4, or TLR9 ligand or combinations thereof), and one or more cellular adhesion agent can be used in combination (e.g., in a same formulation or separately) with an anti-canine OX40 activating antibody provided herein. In some embodiments, an OX40 activating antibody, one or more TLR ligand, one or more cellular adhesion agent, or the combination thereof can be complexed with the cationic liposome. In some embodiments, the one or more TLR ligand includes, but is not limited to, a TLR3 agonist, a TLR4 agonist, a TLR9 agonist, or a combination thereof. In some embodiments, the one or more TLR ligand include, but are not limited to, polyIC, and non-coding plasmid DNA or CpG complexes. In yet other embodiments, an anti-canine OX40 activating antibody can be administered before, simultaneously with, or after another a therapeutic such as an anti-microbial agent (e.g., an anti-viral, an anti-bacterial, an anti-fungal, or an anti-parasitic agent) or immune enhancing composition. In accordance with these embodiments, certain treatment regimens for treating a canine afflicted with a cancer or an immunosuppressant condition are described in more detail below.

In certain embodiments, a dosing regimen described herein can include administering an activating anti-canine OX40 antibody to a dog as described herein as a direct injection (e.g., into a solid tumor). In other embodiments, a dosing regimen can include, but is not limited to, administering an activating anti-canine OX40 antibody systemically (e.g., using parenteral administration). In certain embodiments, a dosing regimen can further include, but is not limited to, administering additional active agents (as described herein). In accordance with these embodiments, certain routes of administration suitable for delivering an activating anti-canine OX40 antibody composition are described in more detail below.

### I. Isolated Activating Anti-Canine OX-40 Antibodies

In some embodiments, an isolated activating anti-canine OX40 antibody is provided. As used herein, the terms “activating anti-canine OX40 antibody” or “activating antibody” can refer to an antibody capable of binding to and activating a canine OX40 polypeptide (e.g., an OX40 polypeptide expressed on a canine cell surface) to induce one or more activities of OX40. In certain embodiments, canine OX40 polypeptides can include an amino acid sequence having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NOs: 1 and 19. In some embodiments, antibody clones disclosed herein can bind to one or more epitope on a canine OX40 polypeptide having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to a polypeptide represented by SEQ ID NOs: 1 and 19 wherein binding the one or more epitopes activates the canine OX40 polypeptide.

SEQ ID NO: 1:
MEHNCFGNTYPKDGKCCNDCPPGYGMESRCSRSHDTKCHQCPSGFYNEAT
NYEPCKPCTQCNQRSGSEPKRRCTPTQDTICSCKPGTEPRDGYKRGVDCA
PCPPGHFSPGDDQACKPWTKLYLMKRRTMQPASKSSDAVCEDRSLPATLP
WETQSPLTRPPTPQPTMAWPRTSQGPFTPPTEPPRGPQGS
SEQ ID NO: 19:
MRMFVESLRLSGPHSALLLLGLVLGAVAEHNCFGNTYPKDGKCCNDCPPG
YGMESRCSRSHDTKCHQCPSGFYNEATNYEPCKPCTQCNQRSGSEPKRRC
TPTQDTICSCKPGTEPRDGYKRGVDCAPCPPGHFSPGDDQACKPWTKLYL
MKRRTMQPASKSSDAVCEDRSLPATLPWETQSPLTRPPTPQPTMAWPRTS
QGPFTPPTEPPRGPQLAAVLGLGLGLLAPVAAALALLLHHRAWRLPPGGN
SFRTPIQEEHADANSTLAKIGS

In other embodiments, isolated activating anti-canine OX40 antibodies described herein can specifically bind to one or more epitope on a polypeptide having an amino acid sequence having SEQ ID NOs: 1 or 19.

As known in the art, a typical antibody molecule includes a heavy chain variable region (VH) and a light chain variable region (VL), which are usually included in antigen binding. The VH and VL regions can be further subdivided into regions of hypervariability, also known as “complementarity determining regions” (“CDR”), interspersed with regions that are more conserved, which are referred to as “framework regions” (“FR”). Each VH and VL is typically composed of three CDRs and four FRs, arranged from amino-terminus to carboxyl-terminus in the following order: FR1, CDR1, FR2, CDR2, FR3, CDR3, FR4. The extent of the framework region and CDRs can be precisely identified using methodology known in the art,

In some embodiments, provided herein are representative OX40 activating antibodies: monoclonal OX40 antibody clone 7E10F3 and monoclonal OX40 antibody clone CAF7 both for activating canine OX40. In certain embodiments, activating antibodies can include a V_{H} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to the V_{H} region of monoclonal OX40 antibody clone 7E10F3 or monoclonal OX40 antibody clone CAF7. In certain embodiments, activating antibodies can include a V_{L} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to the V_{L} region of monoclonal OX40 antibody clone 7E10F3 or monoclonal OX40 antibody clone CAF7.

In certain exemplary embodiments, the V_{H} and V_{L} domains of monoclonal OX40 antibody clone CAF7 are provided herein below as SEQ ID NO: 2 and SEQ ID NO: 3, respectively. In this table, CDRs are indicated in bold. Accordingly, isolated activating anti-canine OX40 antibodies provided herein can have a V_{H} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 2. In certain embodiments, activating antibodies can have a V_{L} region having 85% or more (e.g., 90%, 95%, 98%, 99% or more) sequence identity to SEQ ID NO: 3.

In further embodiments, isolated activating anti-canine OX40 antibodies can have a CDR region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to a corresponding CDR region in monoclonal OX40 antibody clone 7E10F3 or monoclonal OX40 antibody clone CAF7. For example, isolated activating anti-canine OX40 antibody can have a CDR_{H1} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 4, a CDR_{H2} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 5, CDR_{H3} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 6, a CDR_{L1} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 7, a CDR_{L2} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 8, and/or a CDR_{L3} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to SEQ ID NO: 9.

TABLE 1
Exemplary V_{H} and V_{L} regions of monoclonal OX40
antibody clone CAF7

| | | SEQ |
|---|---|---|
| | | ID |
| Name | Sequence | NO: |
| V_{H} | DVLLVESGGDLVKPGGTLRLSCVASGFPFSNFNMGWVRQ | 2 |
| | APGKGLQWVAWIHGSGMTTRYADDVTGRFTISRDNAKD | |
| | TLYLEMDSLRLEDTAKYYCARDLDDAYLGPNWFSYWGQ | |
| | GTLVIVSSASTTAPSVFPLAPSCGSTSGSTVALACLVSG | |
| | YFPEP | |
| V_{L} | DIVMTQAPPSLSLSPGEPASISCKASQSLLHSNGNTYLY | 3 |
| | WFRQKPGQSPEGLIYKVSDRFTGVSDRFSGSGSGTDFTL | |
| | RISRVEADDAGVYYCGQNLQLPYSFSQGTKLEIK | |

TABLE 2
Exemplary CDR regions of monoclonal OX40 antibody
clone CAF7

| | | SEQ ID |
|---|---|---|
| CDR | Sequence | NO: |
| CDR_{H1} | GFPFSNFNMG | 4 |
| CDR_{H2} | IHGSGMTTRYADDVT | 5 |
| | G | |
| CDR_{H3} | DLDDAYLGPNWFSY | 6 |
| CDR_{L1} | KASQSLLHSNGNTYLY | 7 |
| CDR_{L2} | KVSDRFT | 8 |
| CDR_{L3} | GQNLQLPYS | 9 |

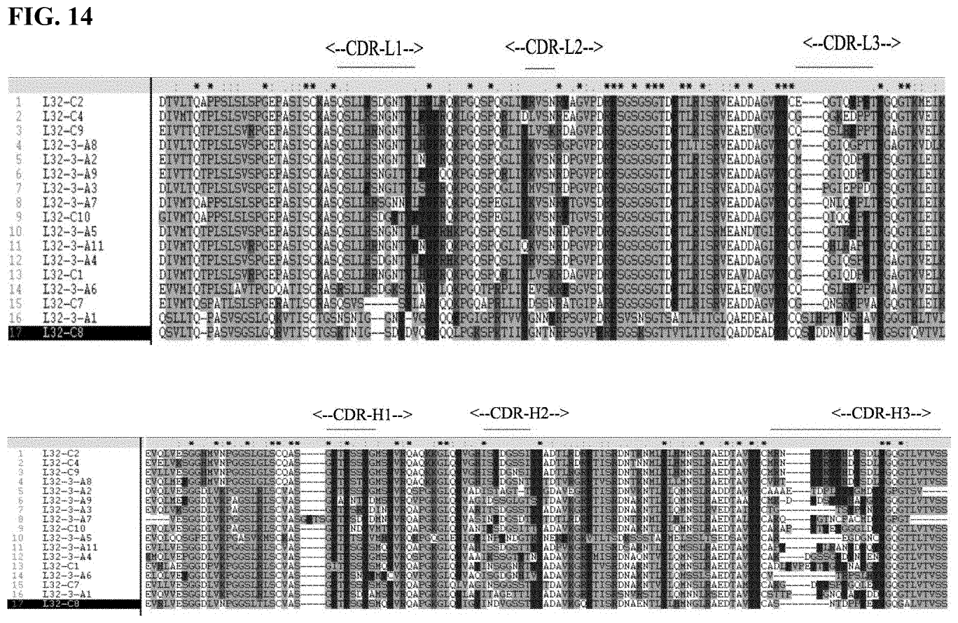

In other embodiments, additional VH regions and VL regions of representative OX40 antigen binding fragments (Fab) are provided herein in FIG. 14. CDR regions are labeled therein. Accordingly, in additional embodiments, isolated activating anti-canine OX40 antibodies can have a VH region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to any of the VH regions provided in FIG. 14. In certain embodiments, activating antibodies can have a VL region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to any of the VL regions provided in FIG. 14. In addition, isolated activating anti-canine OX40 antibodies can have at least one CDR region (e.g., CDRH1, CDRH2, CDRH3, CDRL1, CDRL2 or CDRL3) having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to the corresponding CDR region in the antigen binding fragments provided in FIG. 14.

In other embodiments, additional V_{H} regions and V_{L} regions of representative OX40 antigen binding fragments (Fab) are provided herein in FIG. 14. CDR regions are labeled therein. Accordingly, in additional embodiments, isolated activating anti-canine OX40 antibodies can have a V_{H} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to any of the V_{H} regions provided in FIG. 14. In certain embodiments, activating antibodies can have a V_{L} region having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to any of the V_{L} regions provided in FIG. 14. In addition, isolated activating anti-canine OX40 antibodies can have at least one CDR region (e.g., CDR_{H1}, CDR_{H2}, CDR_{H3}, CDR_{L1}, CDR_{L2} or CDR_{L3}) having about 80%-85%, or more, for example about 86%, about 87%, about 88%, about 89%, about 90%, about 91%, about 92%, about 93%, about 94%, about 95%, about 96%, about 97%, about 98%, about 99% or more or 100% sequence identity to the corresponding CDR region in the antigen binding fragments provided in FIG. 14.

In some embodiments, the heavy chain of any of the anti-OX40 activating antibodies as described herein can further include a heavy chain constant region (CH) or a portion thereof (e.g., CH1, CH2, CH3, or a combination thereof). In accordance with some embodiments disclosed herein, a heavy chain constant region can be of any suitable origin. In some embodiments, the light chain of activating anti-canine OX40 antibodies can further include a light chain constant region (CL), which can be any CL known in the art. In some embodiments, the CL can be a kappa light chain. In some embodiments, the CL can be a lambda light chain. Antibody heavy and light chain constant regions are well known in the art, e.g., those provided in the IMGT database (www.imgt.org) or at www.vbase2.org/vbstat.php., both of which are incorporated by reference herein. In addition, representative CH (CH123) and CL (CK) domains are provided herein as SEQ ID NOs: 10 and 11 (Table 3).

TABLE 3
Constant Regions of monoclonal OX40 antibody
clone CAF7

| Con- | | SEQ |
|---|---|---|
| stant | | ID |
| Regions | Sequence | NO: |
| CH123 | VTVSWNSGSLTSGVHTFPSVLQSSGLYSLSSMVTVPSS | 10 |
| | RWPSETFTCNVAHPASKTKVDKPVPKRENGRVPRPPD | |
| | CPKCPAPEMLGGPSVFIFPPKPKDTLLIARTPEVTCVVV | |
| | DLDPEDPEVQISWFVDGKQMQTAKTQPREEQFNGTYR | |
| | VVSVLPIGHQDWLKGKQFTCKVNNKALPSPIERTISKA | |
| | RGQAHQPSVYVLPPSREELSKNTVSLTCLIKDFFPPDID | |
| | VEWQSNGQQEPESKYRTTPPQLDEDGSYFLYSKLSVD | |
| | KSRWQRGDTFICAVMHEALHNHYTQESLSHSPGK | |
| CK | RTDAQPAVYLFQPSPDQLHTGSASVVCLLNSFYPKDIN | 11 |
| | VKWKVDGVIQDTGIQESVTEQDKDSTYSLSSTLTMSST | |
| | EYLSHELYSCEITHKSLPSTLIKSFQRSECQRVD | |

In accordance with these embodiments, an isolated activating anti-canine OX40 antibody can be monoclonal OX40 antibody such as clone 7E10F3 or monoclonal OX40 antibody such as clone CAF7. In certain embodiments, an isolated activating anti-canine OX40 antibody can be a full-length antibody, which contains two heavy chains and two light chains, each including a variable domain and a constant domain. In certain embodiments, an isolated activating anti-canine OX40 antibody can be an antigen binding fragment of a full-length antibody capable of activating OX40 or certain OX40 activities of use to treat cancer. In accordance with these embodiments, an isolated activating anti-canine OX40 antibody or antigen binding fragment thereof described herein can have binding fragments encompassed within the term “antigen-binding fragment” of a full length antibody can include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment, which consists of a V_{H} domain; and (vi) an isolated complementarity determining region (CDR) that retains functionality. In accordance with these embodiments, an OX40 antibody disclosed herein can have two domains of the Fv fragment wherein, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv).

In some embodiments, isolated activating anti-canine OX40 antibodies disclosed herein can be a single chain antibody (scFv). In accordance with some embodiments herein, a scFv antibody can be a V_{H} fragment and a V_{L} fragment, which can be linked via a flexible peptide linker. In some embodiments, a scFv antibody herein can be in the V_{H}→V_{L} orientation (from N-terminus to C-terminus). In some embodiments, a scFv antibody can be in the V_{L}→V_{H} orientation (from N-terminus to C-terminus).

In some embodiments, an isolated activating anti-canine OX40 antibody herein can be monoclonal antibody.

In some embodiments, an isolated activating anti-canine OX40 antibody as described herein can have a suitable binding affinity for the target antigen (e.g., OX40, SEQ ID NOs: 1, 19) or antigenic epitopes thereof. As used herein, “binding affinity” refers to the apparent association constant or K_{A}. The K_{A} is the reciprocal of the dissociation constant (K_{D}), which can be determined according to known methods in the art.

### II. Preparation and Isolation of Activating Anti-Canine OX40 Antibodies

In certain embodiments, antibodies capable of binding and activating canine OX40 as described herein can be made by any method known in the art. See, for example, Harlow and Lane, (1998) Antibodies: A Laboratory Manual, Cold Spring Harbor Laboratory, New York. In certain embodiments, monoclonal antibodies can be generated using a conventional hybridoma technology and/or by isolating from an antibody library (e.g., obtained from antisera of an immunized animal). Isolated antibodies can be screened for a specific binding affinity to OX40 (and not to other targets). Once antibodies generated with high binding affinity are identified, the OX40 antibodies can be sequenced and recombinantly expressed, and then administered in an in vivo, in situ, or in vitro setting to analyze the ability of the antibodies to activate OX40.

### Generating Antibodies that Bind OX40

### Hybridoma Technology

In some embodiments, antibodies specific to a target antigen (e.g., OX40 or a CRD thereof) can be made by conventional hybridoma technology. In some embodiments, the full-length target antigen or a fragment thereof, optionally coupled to a carrier protein such as KLH, can be used to immunize a host animal (e.g., a mouse or a canine) for generating antibodies binding to that antigen. In some embodiments, the route and schedule of immunization of the host animal are generally in keeping with established and conventional techniques for antibody stimulation and production, as further described herein. In some embodiments, general techniques for production of mouse anti-canine or canine anti-canine antibodies, are known in the art and are described herein. In some embodiments, it is contemplated that any mammalian subject, including dogs, or antibody producing cells therefrom can be manipulated to serve as the basis for production of mammalian, including canine hybridoma cell lines. In some embodiments, the host animal can be inoculated intraperitoneally, intramuscularly, orally, subcutaneously, intraplantar, and/or intradermally with an amount of immunogen, including as described herein. In some embodiments, immunization of a host animal (e.g., a canine) with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCI, or R1N=C=NR, where R and R1 are different alkyl groups, can yield a population of antibodies for use herein.

In some embodiments, hybridomas can be prepared from the lymphocytes and immortalized myeloma cells using the general somatic cell hybridization techniques known in the art. Available myeloma lines can be used. In some embodiments, the technique that can be used herein can include fusing myeloma cells and lymphoid cells to produce hybridomas. In some embodiments, cell fusion technique, EBV immortalized B cells can be used to produce the OX40 monoclonal antibodies described herein. In accordance with these embodiments, hybridomas can be expanded and subcloned, and supernatants can be assayed for anti-immunogen activity by conventional immunoassay procedures (e.g., radioimmunoassay, enzyme immunoassay, or fluorescence immunoassay).

In certain embodiments, hybridomas that can be used as source of antibodies herein can encompass all derivatives, progeny cells of the parent hybridomas that produce monoclonal antibodies capable of activating OX40. In some embodiments, hybridomas herein that can produce such antibodies can be grown in vitro or in vivo using known procedures.

In some embodiments, monoclonal antibodies can be isolated from the culture media or body fluids, by conventional immunoglobulin purification procedures such as ammonium sulfate precipitation, gel electrophoresis, dialysis, chromatography, and ultrafiltration, and the like. In some embodiments, undesired activity, if present, can be removed by, for example but not limited to, running the preparation over adsorbents made of the immunogen attached to a solid phase and eluting or releasing the desired antibodies off the immunogen.

### Isolation from an Antibody Library

In certain embodiments, antibodies capable of binding and activating the target antigens (e.g., canine OX40) as described herein can be isolated from an antibody library generated by immunizing an animal and collecting antisera from the animal. This generates a library of unique antibodies or antibody components that can be used to identify antibodies that bind to a specific target antigen (e.g., OX40 in this case) following routine selection processes as known in the art. In some embodiments, an antibody library can be probed with the target antigen or a fragment thereof and members of the library that are capable of binding to the target antigen can be isolated, typically by retention on a support. In some embodiments, a screening process herein can be performed by multiple rounds (e.g., including both positive and negative selections) to enrich the pool of antibodies capable of binding to the target antigen. In other embodiments, individual clones of the enriched pool can then be isolated and further characterized to identify those having desired binding activity and biological activity. In some embodiments, sequences of the heavy chain and light chain variable domains can also be determined via conventional methodology. There are many routine methods known in the art to identify and isolate antibodies capable of binding to the target antigens described herein, including phage display, yeast display, ribosomal display, or mammalian display technology.

In certain embodiments, phage displays herein can use a covalent linkage to bind the protein (e.g., antibody) component to a bacteriophage coat protein. In some embodiments, the linkage can result from translation of a nucleic acid encoding the antibody component fused to the coat protein. In some embodiments, the linkage can include a flexible peptide linker, a protease site, or an amino acid incorporated as a result of suppression of a stop codon. In some embodiments, a bacteriophage displaying the protein component can be grown and harvested using standard phage preparatory methods, (e.g., PEG precipitation from growth media). In some embodiments, after selection of individual display phages, the nucleic acid encoding the selected protein components can be isolated from cells infected with the selected phages or from the phage themselves, after amplification. In some embodiments, individual colonies or plaques can be selected, and then the nucleic acid can be isolated and sequenced.

### Identifying Antibodies or Antigen Binding Proteins Having a Specific Binding Affinity for Canine OX40 to Activate OX40

In certain methods and procedures described above, antibodies capable of binding to OX40 can be prepared and isolated. In certain embodiments, after antibodies are isolated from the procedures described above for binding to the target antigen, each isolated library member can be tested for its ability to bind to a non-target molecule to evaluate its binding specificity. Examples of non-target molecules include, but are not limited to, streptavidin on magnetic beads, blocking agents such as bovine serum albumin, non-fat bovine milk, soy protein, any capturing or target immobilizing monoclonal antibody, or non-transfected cells which do not express the target. In some embodiments, a high-throughput ELISA screen can be used to obtain the data. In some embodiments, an ELISA screen can also be used to obtain quantitative data for binding of each library member to the target as well as for cross species reactivity to related targets or subunits of the target antigen and also under different condition such as pH 6 or pH 7.5. In accordance with some embodiments herein, non-target and target binding data can be compared (e.g., using a computer and software) to identify library members that specifically bind to the target.

In accordance with some embodiments herein, after selecting candidate library members that bind to a target, each candidate library member can be further analyzed, e.g., to further characterize its binding properties for the target, e.g., canine OX40. In some embodiments, each candidate library member can be subjected to one or more secondary screening assays. In some embodiments, the assay can be for a binding property, a catalytic property, an inhibitory property, a physiological property (e.g., cytotoxicity, renal clearance, or immunogenicity), a structural property (e.g., stability, conformation, oligomerization state) or another functional property. In some embodiments, the same assay can be used repeatedly, but with varying conditions, e.g., to determine pH, ionic, or thermal sensitivities.

In some embodiments, assays herein can use a display library member directly, a recombinant polypeptide produced from the nucleic acid encoding the selected polypeptide, or a synthetic peptide synthesized based on the sequence of the selected polypeptide. In some embodiments, selected Fabs can be evaluated or can be modified and produced as intact IgG proteins.

In some embodiments, binding proteins can be evaluated using an ELISA assay. In accordance with some embodiments herein, each protein can be contacted to a microtiter plate whose bottom surface has been coated with the target, e.g., a limiting amount of the target. In accordance with some embodiments herein, plate can be washed with buffer to remove non-specifically bound polypeptides. In accordance with these embodiments herein, the amount of the binding protein bound to the target on the plate can be determined by probing the plate with an antibody that can recognize the binding protein, e.g., a tag or constant portion of the binding protein. In accordance with some embodiments herein, the antibody can be linked to a detection system (e.g., an enzyme such as alkaline phosphatase or horse radish peroxidase (HRP) which produces a colorimetric product when appropriate substrates are provided).

In some embodiments, binding proteins can be screened for ability to bind to cells which transiently or stably express and display the target of interest on the cell surface. In some embodiments, OX40 binding proteins herein can be fluorescently labeled and binding to OX40 in the presence or absence of antagonistic antibody can be detected by a change in fluorescence intensity using flow cytometry e.g., a FACS machine.

In certain embodiments, at any time during the antibody generation process, one or more positive antibodies are identified. In certain embodiments, the methods further include sequencing the positive antibodies and expressing the sequence in a recombinant microorganism, as described in section 3.

### Polynucleotides and Host Cells/Recombinant Technology

In certain embodiments, a polynucleotide is provided herein, the polynucleotide encoding any activating anti-canine OX40 antibody described herein, or a fragment thereof. For example, the polynucleotide can encode a full antibody (e.g., a single chain construct), or just the heavy or light chain (or variable region alone) of the antibody. In further embodiments, the polynucleotide can further include a vector for expressing the encoded antibody. In addition, a host cell is provided having a polynucleotide described herein according to certain aspects of the invention. Any of the nucleic acids encoding the heavy chain, the light chain, or both of an OX-40 antibody as described herein, vectors (e.g., expression vectors) containing such; and host cells having the vectors are within the scope of the present disclosure.

Polynucleotides, vectors, and host cells can be used to prepare an activating anti-canine OX40 antibody using recombinant technology, as exemplified below.

In certain embodiments, nucleic acids encoding the heavy and light chain of an OX40 antibody as described herein can be cloned into one expression vector, each nucleotide sequence being in operable linkage to a suitable promoter. In some embodiments, each of the nucleotide sequences encoding the heavy chain and light chain is in operable linkage to a distinct promoter. In some embodiments, nucleotide sequences encoding the heavy chain and the light chain can be in operable linkage with a single promoter, such that both heavy and light chains are expressed from the same promoter. In some embodiments, when necessary, an internal ribosomal entry site (IRES) can be inserted between the heavy chain and light chain encoding sequences.

In some embodiments, nucleotide sequences encoding the two chains of the antibody can be cloned into two vectors, which can be introduced into the same or different cells. In some embodiments, when the two chains are expressed in different cells, each of them can be isolated from the host cells expressing such and the isolated heavy chains and light chains can be mixed and incubated under suitable conditions allowing for the formation of the antibody.

In certain embodiments, a nucleic acid sequence encoding one or all chains of an antibody can be cloned into a suitable expression vector in operable linkage with a suitable promoter using methods known in the art. In some embodiments, the nucleotide sequence and vector can be contacted, under suitable conditions, with a restriction enzyme to create complementary ends on each molecule that can pair with each other and be joined together with a ligase. In some embodiments, synthetic nucleic acid linkers can be ligated to the termini of a gene. These synthetic linkers contain nucleic acid sequences that correspond to a particular restriction site in the vector. In some embodiments, selection of expression vectors/promoter can depend on the type of host cells for use in producing the antibodies.

In certain embodiments, the expression and/or production of the antibodies in the host cell can be promoted by expressing a leader peptide ahead of the variable region of the heavy or light chain of the antibody. This leader peptide can be optimized for expression in a certain host cell (e.g., E. coli). One representative sequence that can be used as a leader peptide includes, but is not limited to, the sequence of MRAWIFFLLCLAGRALAAPLA (SEQ ID NO: 12) or fragment thereof. Other leader sequences that are the same length, shorter and longer are known in the art and contemplated of use herein.

In some embodiments, genetically engineered antibodies such as single-chain antibodies can be produced via, e.g., conventional recombinant technology or any methods known in the art. In some embodiments, DNA encoding monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA can be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA can then be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some embodiments, genetically engineered antibodies, such as chimeric or hybrid antibodies; can be prepared that have the binding specificity of a target antigen.

In some embodiments, a single-chain antibody herein can be prepared via recombinant technology by linking a nucleotide sequence coding for a heavy chain variable region and a nucleotide sequence coding for a light chain variable region. In some embodiments, a flexible linker is incorporated between the two variable regions. In some embodiments, techniques described to produce single chain antibodies can be adapted to produce a phage or yeast scFv library and scFv clones specific to OX40 can be identified from the library following routine procedures. In some embodiments, positive clones can be subjected to further screening to identify those that bind to OX40.

In some embodiments, one or more vectors (e.g., expression vectors) having nucleic acids encoding any of the antibodies herein can be introduced into suitable host cells for producing the antibodies. In some embodiments, host cells can be cultured under suitable conditions for expression of the antibody or any polypeptide chain thereof. In some embodiments, antibodies or polypeptide chains thereof can be recovered by the cultured cells (e.g., from the cells or the culture supernatant) via a conventional method, e.g., affinity purification. In some embodiments, polypeptide chains of the antibody herein can be incubated under suitable conditions for a suitable period of time allowing for production of the antibody.

In some embodiments, methods for preparing an antibody described herein can include a recombinant expression vector that encodes both the heavy chain and the light chain of an OX-40 antibody, as also described herein. In some embodiments, a recombinant expression vector can be introduced into a suitable host cell (e.g., a dhfr-CHO cell) by a conventional method, e.g., calcium phosphate-mediated transfection. In some embodiments, positive transformant host cells can be selected and cultured under suitable conditions allowing for the expression of the two polypeptide chains that form the antibody, which can be recovered from the cells or from the culture medium. In some embodiments, the two chains recovered from the host cells can be incubated under suitable conditions allowing for the formation of the antibody.

In certain embodiments, two recombinant expression vectors are provided, one encoding the heavy chain of the OX40 antibody and the other encoding the light chain of the OX40 antibody. In some embodiments, both the recombinant expression vectors can be introduced into a suitable host cell (e.g., dhfr-CHO cell) by a conventional method, e.g., calcium phosphate-mediated transfection. In some embodiments, each of the expression vectors can be introduced into a suitable host cell. In some embodiments, positive transformants can be selected and cultured under suitable conditions allowing for the expression of the polypeptide chains of the antibody. In some embodiments, when the two expression vectors are introduced into the same host cells, the antibody produced therein can be recovered from the host cells or from the culture medium. In some embodiments, the polypeptide chains can be recovered from the host cells or from the culture medium and then incubated under suitable conditions allowing for formation of the antibody. In some embodiments, when the two expression vectors are introduced into different host cells, each of them can be recovered from the corresponding host cells or from the corresponding culture media. In some embodiments, two polypeptide chains can then be incubated under suitable conditions for formation of the antibody.

In certain embodiments, standard molecular biology techniques can be used to prepare the recombinant expression vector, transfect the host cells, select for transformants, culture the host cells and recovery of the antibodies from the culture medium. In some embodiments, some antibodies can be isolated by affinity chromatography with a Protein A or Protein G coupled matrix.

### Characterization and Identification of Activating Anti-Canine Ox40 Antibodies

In certain embodiments, activating anti-canine OX-40 antibodies herein can be characterized using methods known in the art, whereby activation or an increase of OX-40 biological activity is detected and/or measured. In certain embodiments, the increase of OX40 biological activity can include an increase of T cell activity, proliferation or survival; and/or a decrease in regulatory T cell (Treg) activity, proliferation or survival.

In some embodiments, methods of identifying an activating antibody of OX40 are provided, the method involving culturing a host cell described above under conditions allowing for expression of the antibody (or heavy or light chain thereof) that binds OX40, harvesting the antibody produced from the cell culture, and screening for activity of OX40.

In certain embodiments, OX40 activity induced by an antibody disclosed herein can be screened by detecting an increase in non-regulatory T cell activation, T-cell proliferation or improved/prolonged survival, upregulation of other activation markers in an organism administered an OX40 antibody provided herein. In additional embodiments, OX40 activity can be screened by detecting a decrease in regulator T cell activity, proliferation or survival in an organism following administration of an OX40 antibody provided herein. In another embodiment, multiple in vitro assays, can be used to establish the activity of a targeted isolated antibody disclosed.

In additional embodiments, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, competition assays, gene fragment expression assays, and synthetic peptide-based assays. In some embodiments, epitope mapping can be used to determine the sequence, to which an antibody binds.

### Therapeutic Applications of Activating Anti-Canine OX40 Antibodies

In some embodiments, OX40 activation can act to increase innate immunity through at least two mechanisms. In accordance with these embodiments, stimulation of OX40 on non-regulatory T cells can lead to their upregulation and proliferation as well as enhance their survival. In other embodiments, stimulation of OX40 on regulatory T cells (Tregs) can lead to reduction or depletion of these Treg cells. In accordance with these embodiments, because for example, regulatory T cells act to breakdown the naturally-occurring immune system, depleting their population can lead to an overall upregulation in immune response. This dual action can be exploited to boost overall immunity and treat various immunosuppressed conditions using activating OX40 antibodies disclosed herein.

In some embodiments, activating anti-canine OX40 antibodies disclosed herein can be used for therapeutic, diagnostic, and/or research purposes in a canine system, all of which are within the scope of the present disclosure. In certain embodiments these therapeutic uses can include a variety of dosing and treatment regimens that collectively deliver an activating anti-canine OX40 antibody to a subject in need thereof, optionally alongside other agents which may work synergistically with the antibody to improve the condition. In some embodiments, methods of treating a solid tumor cancer can include administering an activating anti-canine OX40 antibody directly into a tumor (e.g., via intratumoral injection) of a dog every week (e.g., for 4 weeks) or every month (e.g., for 6 months). In certain embodiments, the dog can also be administered an anti-cancer therapeutic (such as radiation therapy or a pharmaceutical agent). In some embodiments, the dog can be treated with radiation therapy for one or more days prior to or after administering compositions containing activating anti-canine OX40 antibodies. In some embodiments, the dog can be administered a secondary treatment either at the same time as the activating OX40 antibody or about 1, about 2, about 3, about 4, or about 5 days before, or about 1, about 2, about 3, about 4, or about 5 days after each dose of the activating OX40 antibody. In some embodiments, the secondary treatment can include a non-specific immune response stimulator (e.g., containing a TLR ligand and excipients suitable for the delivery of the TLR ligand to a target). Additional secondary treatments that can be combined with an activating OX40 antibody can include, but are not limited to antimicrobial agents such as, anti-virals, anti-bacterials, anti-fungals, and anti-protazoic agents.

### Pharmaceutical Compositions

In certain embodiments, pharmaceutical compositions are provided herein. The pharmaceutical composition can contain a pharmaceutically acceptable carrier combined with an activating anti-canine OX40 antibody as described herein and/or one or more encoding polynucleotides or vector thereof that can facilitate the expression of the anti-canine OX40 antibody in a canine. Pharmaceutically acceptable excipients (carriers) are well known in the art.

In certain embodiments, the pharmaceutical compositions can further include a non-specific innate immune response stimulator mixture or composition (having, for example, one or more TLR ligand). In accordance with these embodiments, the non-specific innate immune response stimulator can elicit both a cell-mediated immune response and a humoral immune response. In certain embodiments, the non-specific innate immune response stimulator is an immunogenic composition containing (a) one or more cationic liposomes where the one or more cationic liposomes contain a mixture of cationic lipids and non-charged lipids, (b) a mixture of toll like receptor 3 (TLR3) and toll like receptor 9 (TLR9) ligands, and (c) one or more cellular adhesion agent. In certain embodiments the cationic liposomes can contain cationic lipids and cholesterol. In certain embodiments, the TLR3 ligand provided in this immunogenic composition can be polyinosinic-polycytidylic acid. In other embodiments, the TLR9 ligand can be a non-coding plasmid DNA or a CpG oligo. In certain embodiments, the cellular adhesion agent can be carboxymethylcellulose, chitosan, polyglycol, hyaluronan or a combination of any thereof. In certain embodiments, the immunogen composition can include about 1.0% to about 20.0% (v/v) of the cellular adhesion agent (e.g., carboxymethylcellulose). In additional embodiments, the immunogen composition can include from about 100 μg of the TLR3 and TLR9 ligands per 1 mL of a 10 mM cationic liposome concentration. Exemplary non-specific innate immune response stimulators that can be combined with the activating OX40 antibodies in pharmaceutical compositions provided herein and for uses disclosed herein are described in U.S. Pat. No. 10,512,687, the entire disclosure of which is incorporated by reference for all purposes.

In other embodiments, pharmaceutical compositions described herein can further include an anti-microbial agent, a chemotherapeutic agent, and/or an anti-PD-1 antibody. In accordance with these embodiments, the anti-microbial agent can, in an example, be an anti-viral, bactericidal agent, anti-fungal, or anti-bacterial agent. For example, the anti-microbial agent can be an anti-bacterial agent (antibiotic) such as doxycycline or another antibiotic such as a general antibiotic.

In certain embodiments, pharmaceutical composition described herein specifically exclude agents that inhibit or suppress OX40 activity (e.g., an OX40 inhibiting antibody).

Pharmaceutically acceptable carriers or excipients suitable for the compositions described herein are well known to one of skill in the art of use for preserving and delivering antibodies or antibody fragments to a dog.

In certain embodiments, the pharmaceutical compositions to be used in the present methods can include pharmaceutically acceptable carriers, excipients, or stabilizers in the form of lyophilized formulations or aqueous solutions. In some embodiments, acceptable carriers, excipients, or stabilizers are nontoxic to recipients at the dosages and concentrations used, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition described herein can have liposomes containing the antibodies (or the encoding nucleic acids). In some embodiments, liposomes for use herein can be generated by the reverse phase evaporation method with a lipid composition having phosphatidylcholine, cholesterol and PEG-derivatized phosphatidylethanolamine (PEG-PE). In some embodiments, liposomes for use herein can be extruded through filters of defined pore size to yield liposomes with the desired diameter.

In some embodiments, antibodies, or the encoding nucleic acid(s) herein, can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other embodiments, the pharmaceutical compositions described herein can be formulated in sustained-release format. In some embodiments, pharmaceutical compositions herein to be used for in vivo administration must be sterile. In some embodiments, this can be readily accomplished by, for example, filtration through sterile filtration membranes. In some embodiments, therapeutic antibody compositions can be placed into a container having a sterile access port, for example, an intravenous solution bag or vial having a stopper pierceable by a hypodermic injection needle. In certain embodiments, pharmaceutical compositions described herein can be in unit dosage forms such as tablets, pills, capsules, powders, granules, solutions or suspensions, or suppositories, for oral, parenteral, or rectal administration, or administration by inhalation or insufflation. In some embodiments, emulsion compositions herein can be those prepared by mixing an antibody with Intralipid™ or the components thereof (soybean oil, egg phospholipids, glycerol and water).

In some embodiments, pharmaceutical compositions disclosed herein for inhalation or intranasal administration include solutions and suspensions in pharmaceutically acceptable, aqueous or organic solvents, or mixtures thereof, and powders. In some embodiments, liquid or solid compositions disclosed herein can contain suitable pharmaceutically acceptable excipients as set out above. In other embodiments, the compositions can be administered by the oral or nasal respiratory route for local or systemic effect.

In certain embodiments, compositions can be in sterile pharmaceutically acceptable solvents can be nebulized by use of gases. In some embodiments, nebulized solutions disclosed herein can be breathed directly from the nebulizing device or the nebulizing device can be attached to a face mask, tent or intermittent positive pressure breathing machine. In some embodiments, solution, suspension, or powder compositions herein can be administered, preferably orally or nasally, from devices which deliver the formulation in an appropriate manner.

In some embodiments, concentrations of antibodies disclosed herein can be a pre-determined concentration or a standard concentration. In some embodiments, the antibody can be in a concentration of about 1-1000 mg/ml, 1-500 mg/ml, 1-250 mg/ml, 1-200 mg/ml, 1-150 mg/ml, 1-100 mg/ml, 1-75 mg/ml, or 1-50 mg/ml. In some embodiments, the antibody is formulated to a concentration of about 30 mg/ml. In some embodiments, the antibody is lyophilized. In some embodiments, the antibody is diluted in a suitable solution to a suitable concentration prior to administration (e.g., in a therapeutic application described below).

### III(b) Methods of Use—Therapeutic Applications

In certain embodiments, OX40 activation in canines can be used to increase innate immunity to treat canine disease and conditions. In certain embodiments, activation of OX40 can be enhanced by combining with specific innate immune stimulators or other agents. Accordingly, provided herein are methods of increasing innate immunity in a canine in need thereof, including methods for administering to a dog an effective amount of any of the pharmaceutical compositions described herein.

In certain embodiments, activating OX40 antibodies described herein are useful in treating conditions due to cancer or a chronic immunosuppressed condition. For example, in certain solid tumor cancers there can be an imbalance of regulatory T cells and non-regulatory T cells in and around the tumor. In certain embodiments, a solid tumor can have an elevated population of regulatory T cells which, in turn, suppress nearby T cells leading to an overall suppression in the innate anti-tumor immune response. In accordance with these embodiments, administering a composition containing an activating OX40 antibody can, through OX40 activation, deplete the tumor regulatory T cell population, releasing the block of the dog's immune system and promote tumor reduction by allowing the induced immune system to act on the tumor. In certain embodiments, an activating OX40 antibody can enhance the survival and proliferation of non-regulatory T cells which can be a useful outcome for patients afflicted with a chronic infection that leads to immunosuppression. In certain embodiments, certain fungal, bacterial or protozoal infections can cause chronic immunosuppression. In some embodiments, a chronic infection is a family of bacterial infections that cause Rickettsia diseases.

In certain embodiments, the cancer is a solid tumor cancer. In other embodiments, the solid tumor can be a head, neck, lung, breast, liver, or colon tumor. In certain embodiments, the cancer includes, but is not limited to, melanoma, a carcinoma, a sarcoma, or a combination thereof. In some embodiments, administering compositions disclosed herein can be by direct injection into the tumor of the canine. In certain embodiments, the method can include systemic administration of the composition.

In additional embodiments, a method is provided herein of treating immunosuppression in a canine in need thereof, the method including administering to the canine an effective amount of any of the pharmaceutical compositions described herein and treating the immunosuppression. In certain embodiments, the immunosuppression is caused by a chronic viral, a chronic fungal, a chronic bacterial, or a chronic protozoal infection. In some embodiments, the chronic bacterial infection can lead to a series of conditions known collectively as Rickettsia disease. Accordingly, in some embodiments, the methods herein can effectively treat a Rickettsia condition in a canine. In certain embodiments, the method of treating immunosuppression can include administering the pharmaceutical compositions systemically or by localized administration. Additional means of administration are detailed below.

In accordance with these embodiments, combination therapies are contemplated; for example, administering or using at least a secondary treatment at least one of before, during, and after administering a pharmaceutical composition disclosed. In some embodiments, at least the secondary treatment can be directed to treating an immunosuppressive condition. In certain embodiments, the secondary treatment can include an innate immune response stimulator. In accordance with these embodiments, the innate immune stimulator can be a non-specific immunostimulator. In other embodiments, a non-specific innate immune response stimulator can include a composition of one or more of a TLR ligand. In other embodiments, the TLR ligand can include one or more of a TLR3, TLR4 and/or TLR9 ligand. In yet other embodiments, at least one secondary agent can include one or more anti-PD1 antibody, one or more chemotherapeutic agent, one or more antimicrobial or a combination of any thereof. In some embodiments, a secondary agent includes an antibiotic, and the antibiotic includes doxycycline or other selected antibiotic.

In certain embodiments, a dosing regimen for administering an activating anti-canine OX40 antibody can be determined according to methods known in the art. In some embodiments, an activating anti-canine OX40 antibody-containing composition provided herein can be administered to a dog over a series of multiple treatment days in the course of a given treatment regimen. In other embodiments, an activating anti-canine OX40 antibody-containing composition can be administered over the course of 4, 6, or 8 weeks (e.g., using a weekly dosing regimen). In other embodiments, an activating anti-canine OX40 antibody-containing composition can be administered over the course of 4, 6, 8, 10, or 12 months (e.g., using a monthly dosing regimen). In yet other embodiments, as described below, an activating anti-canine OX40 antibody can be administered alongside or close to a secondary treatment suitable for treating a cancer or immunosuppressed condition.

In certain embodiments, methods of treating cancers or immunosuppressed conditions disclosed herein, a secondary treatment can be administered to a canine in a single day or over several days prior to administration of an activating anti-canine OX40 antibody-containing composition. In some embodiments, secondary treatment regimens can include radiation therapy in conjunction with an activating anti-canine OX40 antibody can include administering the radiation therapy to a dog for 1, 2 or 3 days and then administer an activating anti-canine OX40 antibody. In certain embodiments, an activating anti-canine OX40 antibody can be administered over the course of a multiple day/week or month long treatment regimen. In other embodiments, activating anti-canine OX40 antibody-containing compositions can be administered daily, every other day, twice a week, every week, twice a month or once a month or other dosing regimen to a dog depending on the condition to be treated and the severity of the condition such as a chronic infection or early or advanced stage cancer. In other embodiments, a dog can be administered a secondary agent known in the art to treat a condition contemplated herein and secondary agents include, but are not limited to, an immune response stimulator or an anti-microbial. In accordance with these embodiments, these agents can be administered to the dog either simultaneously, prior to, during or after administering an activating OX40 antibody-containing composition. In some embodiments, the secondary active agent can be administered within a few minutes or hours of administering the activating antibody. In accordance with these embodiments, other dosing regimens known in the art can be used for optimal outcome.

In certain embodiments, methods of treating cancer in a canine can further include administering a pharmaceutical composition containing activating OX40 antibodies described herein, as well as a non-specific immune response stimulator including, but not limited to, one or more TLR3 and/or TLR9 ligand as well as using radiation therapy (e.g., SBRT). In some embodiments, activating OX40 antibodies and one or more non-specific immune response stimulator containing a TLR3, TLR4, and/or TLR9 ligand can be prepared in a single formulation. In additional embodiments, an activating OX40 antibody and a non-specific immune response stimulator containing a TLR3, TLR4, and/or TLR9 ligand are prepared in separate formulations (or pharmaceutical compositions) and administered separately, before during or after the radiation therapy. In some embodiments, an activating OX40 antibody and a non-specific immune response stimulator containing a TLR3, TLR4 and/or TLR9 ligand can be administered to a canine in order to treat a solid tumor cancer. In some embodiments, the activating OX40 antibody and the non-specific immune response stimulator containing a TLR3, TLR4, and/or TLR9 ligand can, optionally, be co-injected, or serially injected, directly into a tumor of the canine, alone or before during or after the radiation therapy or other standard therapy known in the art.

In certain embodiments, administration of a treatment via a suitable route, such as intravenous administration, e.g., as a bolus or by continuous infusion over a period of time, can include, intramuscular, intraperitoneal, intracerebrospinal, subcutaneous, intra-articular, intrasynovial, intrathecal, oral, inhalation or topical routes. In some embodiments, pharmaceutical compositions can be administered by conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, compositions disclosed herein can be administered to the subject via injectable depot routes of administration such as using 1-, 3-, or 6-month depot injectable or biodegradable materials and methods. In other embodiments, pharmaceutical compositions described herein can be administered via site-specific or targeted local delivery techniques. Examples of site-specific or targeted local delivery techniques include certain implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application.

In certain embodiments, a canine having a target cancer can be identified by routine medical examination, e.g., laboratory tests, organ functional tests, CT scans, or ultrasounds. In some embodiments, the canine to be treated by the method described herein can have undergone or is subjecting to an anti-cancer therapy, for example, chemotherapy, radiotherapy, immunotherapy, or surgery.

In some embodiments, targeted delivery of therapeutic compositions herein containing an antisense polynucleotide, expression vector, or subgenomic polynucleotides can also be used where an anti-canine OX40 antibody is expressed in vivo. In some embodiments, therapeutic polynucleotides and polypeptides described herein can be delivered using gene delivery vehicles. In other embodiments, the gene delivery vehicle can be of viral or non-viral origin. In some embodiments, expression of such coding sequences can be induced using endogenous mammalian or heterologous promoters and/or enhancers. In other embodiments, expression of the coding sequence can be either constitutive or regulated. In some embodiments, therapeutic polynucleotides and polypeptides described herein can be delivered via viral-based vectors. In other embodiments, non-viral delivery vehicles and methods can also be employed, including, but not limited to, polycationic condensed DNA linked or unlinked to killed adenovirus alone, ligand-linked DNA, eukaryotic cell delivery vehicles cells, and nucleic charge neutralization or fusion with cell membranes. In some embodiments, naked DNA can also be employed. In some embodiments, liposomes can be used herein as gene delivery vehicles.

In some embodiments, treatment efficacy for a target disease/disorder can be assessed by methods well-known in the art. In some embodiments, a method of treatment can include measuring levels of various T cells in to assess depletion and/or activation, expansion as appropriate to induce a non-specific immune response as a result of OX40 antibody treatments alone or in combination with other treatments disclosed herein. In accordance with these embodiments, various T cells measured disclosed herein can include, measuring Treg cells, exhausted T cell, myeloid cell, effector T cell, tumor macrophage, CD3+ T cells, or a combination thereof to determine treatment progress and/or to adjust dosing regimens. In some embodiments, to assess treatment success, methods for measuring Treg-associated gene expression can be performed. In other embodiments, the Treg-associated gene expression can include measuring FoxP3 expression, GATA3 expression, IL10 expression, TGFβ expression, or a combination thereof representative of Treg cell populations. In other embodiments, to assess treatment progression, methods for measuring exhausted T cell-associated gene expression can be used to continue, modify or change treatment regimens. In some embodiments, to measure exhausted T cell-associated gene expression can include measuring CTLA4 expression, IL10 expression, Lag3 expression, PD-1 expression, TGFβ expression, or a combination thereof. In other embodiments, methods can include measuring myeloid cell-associated gene expression to assess the progress of treatment and/or adjust dosing. In accordance with these embodiments, measuring myeloid cell-associated gene expression can include, measuring one or more of CCL2 expression, CD274 expression, CD40 expression, CLEC4C expression, ICOSLG expression, IL8 expression, IL10 expression, IL12 expression, IL6 expression, IL8 expression, MX1 expression, PD-L1 expression, TGFβ expression, TNF expression, or a combination thereof. In other embodiments, methods can include measuring effector T cell-associated gene expression to determine treatment progress and/or adjust dosing. In accordance with these embodiments, the effector T cell-associated gene expression can include assessing one or more of CD27 expression, CD28 expression, CD70 expression, CD8a expression, GZMA expression, GZMB expression, ICOS expression, IFNγ expression, IL2 expression, OX40 expression, PRF1 expression, TNF expression, or a combination thereof. In certain embodiments, measured cell levels or gene expression can be TME-localized. In certain embodiments, combination therapies including OX40 activating antibodies alone or in combination with one or more TLR3, TLR4 and/or TLR9 ligand and/or radiation therapy (e.g., SBRT) resulted in induced innate immunity including, at least one of local depletion of Tregs and reduced Treg-associated gene expression (FoxP3), reduced the density of tumor-associated macrophages, suppressed macrophage-associated gene expression (IL-8), suppressed exhausted T cell-associated gene expression (CTLA4), and induced an increase in circulating IL-7 concentrations compared to dogs treated with radiation (e.g. SBRT) alone. In accordance with these embodiments, cancer progression or remission can be analyzed by any method known in the art to adjust treatment regimens as needed. In some embodiments, treatments disclosed herein include intratumoral treatments. In certain embodiments, radiation therapy can be performed before, during or after, treating with one or more OX40 activating antibodies and/or one or more TLR ligand or non-specific immune modulators disclosed herein

### Kits for Use in Treatment of Diseases

In certain embodiments, kits are provided for storage, transport and use in treating or alleviating a target disease, such an immunosuppressed condition or cancer as described herein. In some embodiments, kits can include one or more containers. In other embodiments, kits disclosed herein contain at least one anti-canine OX40 antibody or composition thereof.

In some embodiments, kits can include instructions for use in accordance with any of the methods described herein. In some embodiments, instructions can be included and can contain a description of administration of the OX40 antibody, and optionally, the second therapeutic agent, to treat, delay the onset, or alleviate a target disease as those described herein. In other embodiments, kits can further include a description of selecting an individual suitable for treatment based on identifying whether that individual has the target disease, e.g., applying the diagnostic method as described herein. In still other embodiments, the instructions can have a description of administering an antibody to an individual at risk of the target disease.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1: Comparison of Effectiveness of OX40 and PD-1 Antibodies for Inhibiting Cancer Growth

In one exemplary method, studies were conducted in a mouse model of breast cancer (PyMT cell line) to compare the relative efficacy of treatment with mouse OX40 (monoclonal antibody clone 7E10F3) versus PD-1 antibodies. Mice (n=5 per group) were implanted with PyMT tumor cells, then treated for 3 consecutive days with intraperitoneal injection of 100 μg each of rat anti-OX40 or rat anti-PD-1 antibodies. The treatment was then repeated one week later. Tumor volume was measured every other day and plotted. Treatment with the OX40 antibody significantly slowed tumor growth compared to PD-1 antibody treatment, which had little impact on tumor growth. These findings suggest that at least for some tumors, OX40 antibody immunotherapy can be more effective than antibodies targeting other T cell checkpoint molecules such as PD-1.

### Example 2: Expression of OX40 on Activated Canine T Cells Detected with Canine OX40 mAb

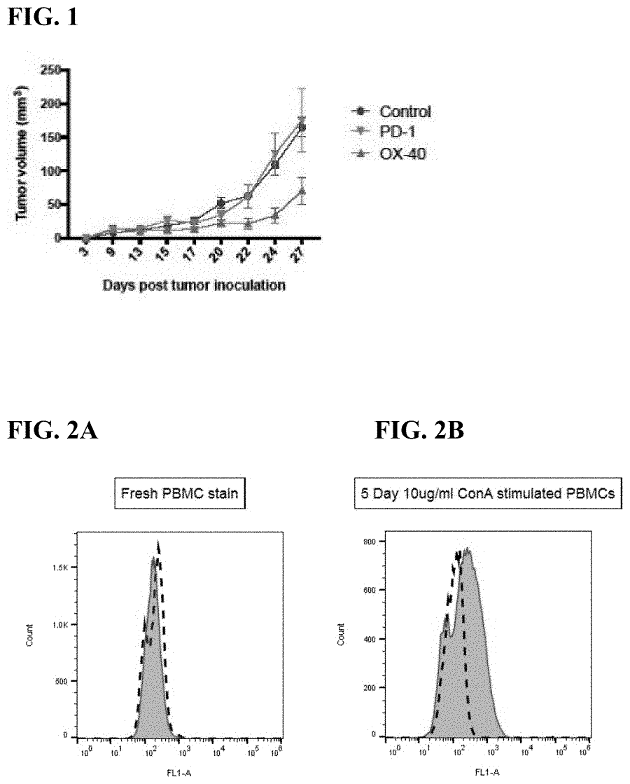

In another exemplary method, peripheral blood from a dog was separated into peripheral blood mononuclear cell (PBMC) cultures, which were then left unstimulated or stimulated with ConA (5 μg/ml) for 5 days. The cells were then immunostained with the murine, anti-canine OX40 mAb (monoclonal antibody clone 7E10F3), and expression of OX40 on T cells assessed using flow cytometry. The resulting cytometry plots are provided in FIG. 2A-2B for unstimulated (2A) or stimulated (2B) samples. The 7E10F3 antibody demonstrated significant upregulation of OX40 expression on activated, but not resting, dog T cells.

### Example 3: Constitutive Expression of OX40 by the Canine B Cell Lymphoma Cell Line CLBL1.

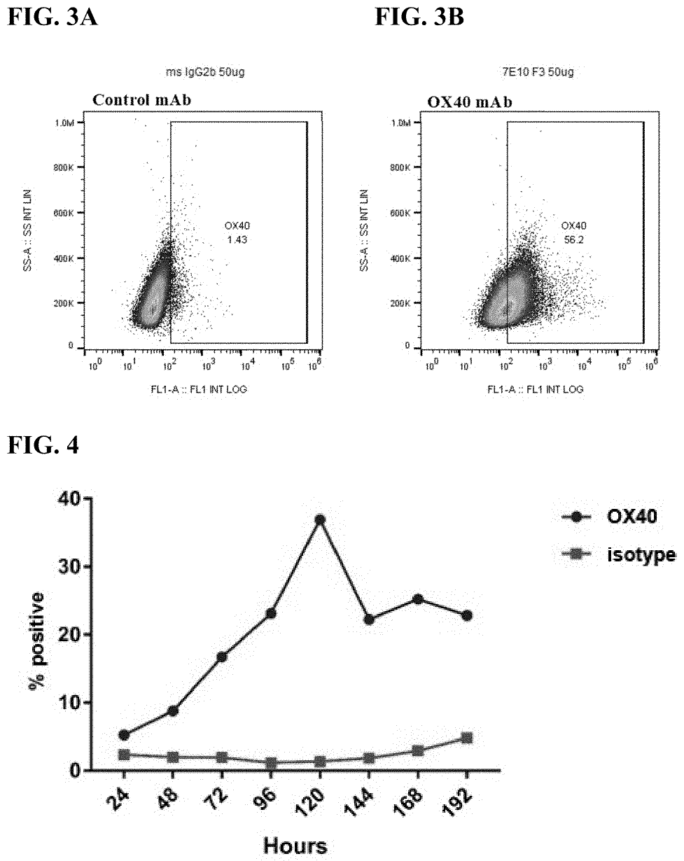

In another exemplary method, a panel of canine tumor cell lines was screened for expression of OX40 using the murine anti-canine OX40 mAb (monoclonal antibody clone 7E10F3), to serve as a tool for investigating expression regulation and for screening new antibodies. The only cell line found to constitutively express OX40 was the canine B cell lymphoma cell line CLBL1, as demonstrated by flow cytometry (FIG. 3).

### Example 4: Kinetics of OX40 Upregulation on Dog T Cells As Assessed Using mAb 7E10F3.

In another exemplary method, canine T cells were activated with ConA (5 μg/ml) and OX40 expression was assessed over time using the murine anti-canine OX40 mAb (monoclonal antibody clone 7E10F3). Expression was upregulated beginning as early as 24 h after stimulation and remained high for at least 192 h (FIG. 4).

### Example 5: Functional Activity of Anti-Canine OX40 mAb 7E10F3

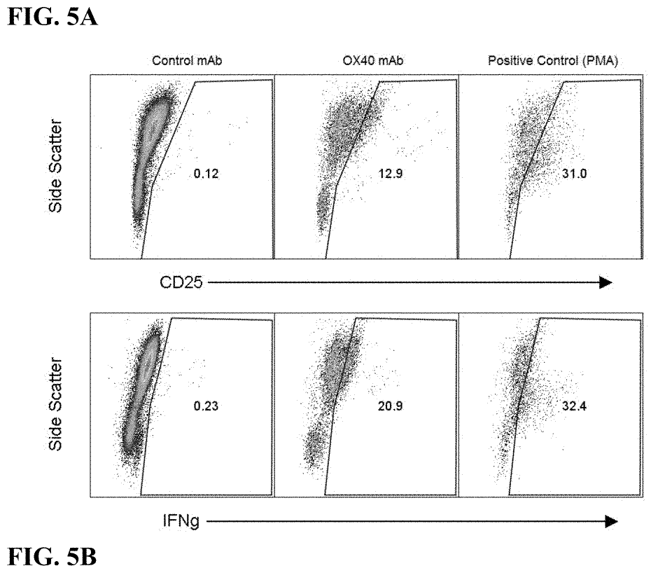

In another exemplary method, canine T cells were activated with a low dose of concanavalin A (ConA) (0.5 μg/ml) and then incubated with irrelevant control mouse mAb, or with mouse anti-canine mAb 7E10F3 (monoclonal antibody clone 7E10F3, 10 μg/ml) for 24h, in the presence of Brefeldin A for the last 12 h of incubation. Positive control cells were activated with PMA and ionomycin combination. Cells were then fixed, and permeabilized and incubated with a cross-reactive anti-bovine INF-g antibody to detect intracellular expression of IFN-g. T cells incubated with the monoclonal antibody clone 7E10F3 OX40 demonstrated significant intracellular expression of IFN-γ, compared to control cells incubated with irrelevant or control antibody (FIG. 5).

### Example 6: Detection of OX40+ T Cells in Tumor Tissues from Canine Melanoma Biopsy

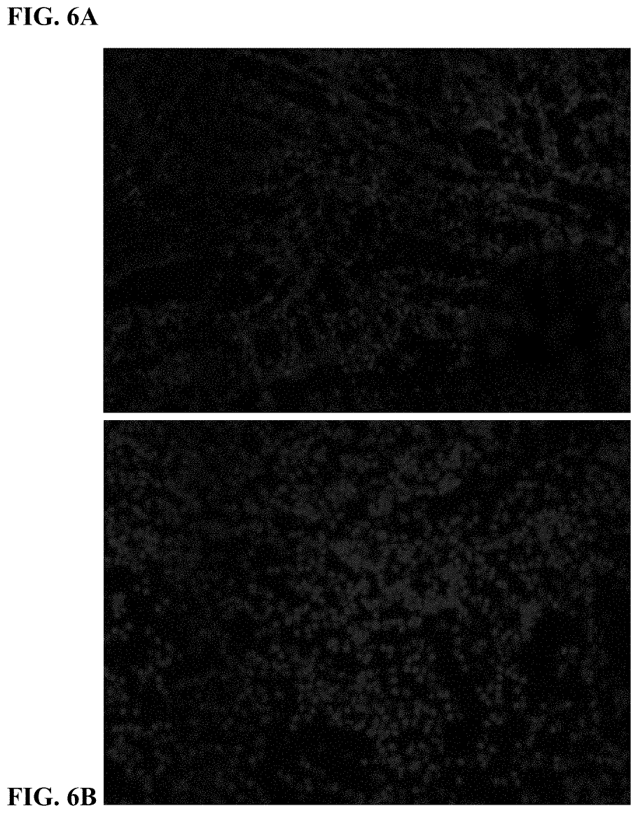

In another exemplary method, using immunohistochemistry, tumor sections were immunostained with monoclonal antibody clone 7E10F3 (FIG. 6A) or with isotype-matched, irrelevant monoclonal antibody (FIG. 6B). Numerous T cells expressing OX40 were detected within tumor tissues.

### Example 7: OX40 Cancer Immunotherapy Study

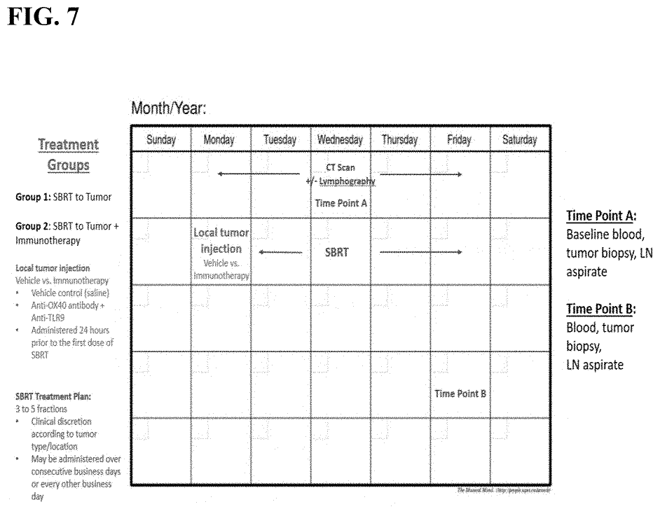

In another exemplary method, a clinical trial in dogs was conducted to assess the impact of OX40 monoclonal antibody direct intratumoral injection on tumor responses to radiation therapy. Dogs with head and neck cancer (either melanoma or squamous cell carcinoma) were randomized to receive stereotaxic body radiation therapy (SBRT) only, or SBRT+ intratumoral injection of OX40 mAb clone 7F10E3 (100-200 μg per tumor, depending on size) as a single injection, in conjunction with intratumoral injection of a TLR3/9 liposomal immunotherapy (100 to 200 ul per tumor) as a single injection, done immediately before RT. Tumor biopsies were collected prior to SBRT and again 7 days later to assess the impact of OX40 mAb injections on tumor immune infiltrates, as assessed by IHC. Table 4 summarizes the study. FIG. 7 depicts the clinical trial design and lists the schedule of treatments for study animals, the RT schedule, and the schedule of biopsy samples.

TABLE 4
Trial Design: Radiation + local immunotherapy study in dogs with head and neck
cancers (melanoma, squamous cell carcinoma)

Study design: Randomized to treatment with 3-fraction SBRT of local tumor (n = 8 dogs)
4 dogs randomized to SBRT plus intratumoral PBS injection
4 dogs randomized to SBRT plus single intra-tumoral OX40 mAb (100-200 ug) + TLR3/9
agonist (100-200 ul) injection
Tumor biopsy prior to Rx and again 7 days later
Tumor tissue analyzed by IHC and Nanostring

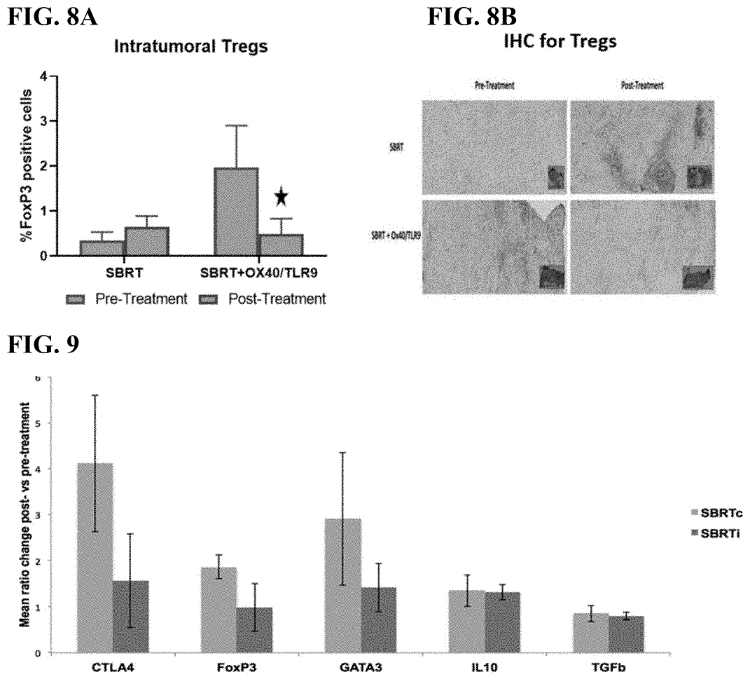

FIG. 8 demonstrates the impact of local OX40 mAb immunotherapy on Treg populations in tumors undergoing SBRT treatment. Tumor biopsies were collected immediately prior to SBRT, and again 7 days after tumor SBRT. There were n=4 animals receiving RT only, and n=4 animals receiving RT+OX40 immunotherapy. Tumor tissues were immunostained for detection of Tregs, using a cross-reactive FoxP3 mAb (FIG. 8, right panel). The density of Tregs was determined using ImageJ software and expressed as percentage of FoxP3+ cells per tumor area (FIG. 8, left panel). Tumors treated with OX40 mAb and RT had significantly fewer Tregs than tumors treated only with RT, indicative of Treg depletion by the OX40 mAb treatment.

### Example 8. Impact of OX40 mAb Treatment on Tumor Expression of Treg Related Genes

In another exemplary method, tumor tissues were collected at day 7 from n=4 dogs treated with SBRT alone (SBRTc) or n=4 dogs treated with SBRT+OX40 immunotherapy (SBRTi). Gene expression was quantitated using Nanostring panels. Expression of CTLA4, FoxP3, and GATA3 was significantly lower in tumors treated with SBRTi than in tumors treated with SBRTc, indicative of modulation of Tregs and their associated genes following OX40 immunotherapy (FIG. 9).

### Example 9. Generation of Anti-OX40 Monoclonal Antibody

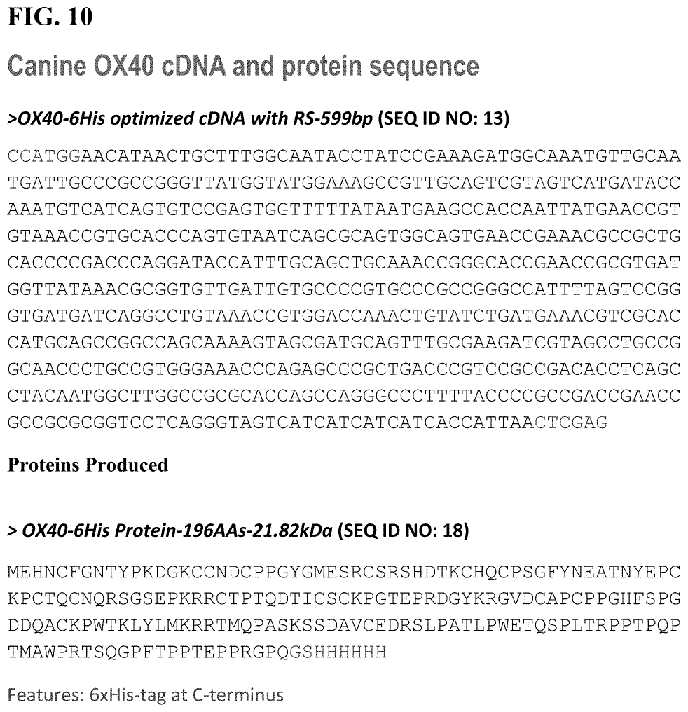

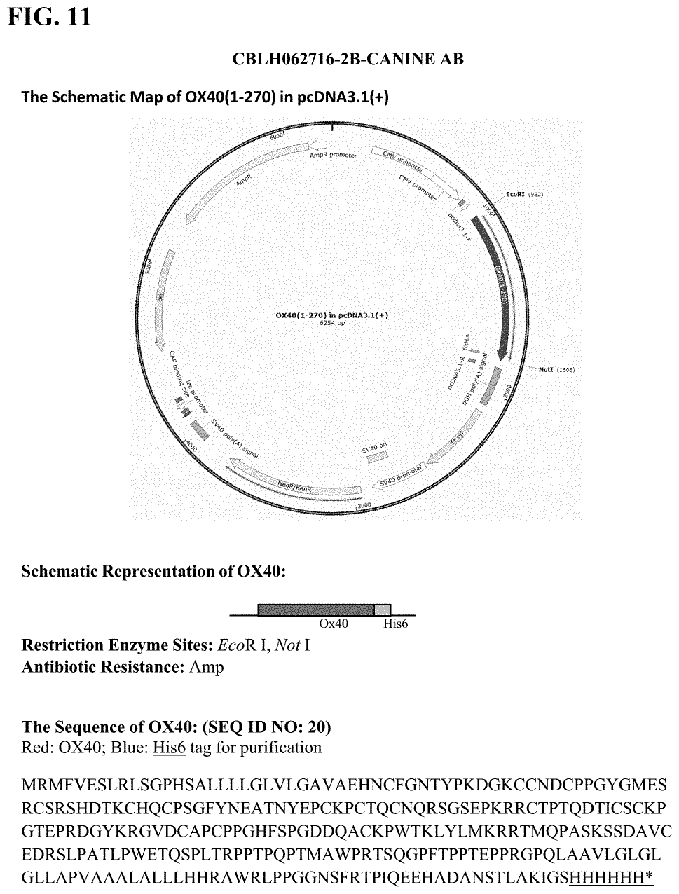

In another exemplary method, a canine OX40 cDNA was synthesized and codon optimized for expression in E. coli. The sequence of the OX40 cDNA is provided herein in Table 5 as SEQ ID NO: 13, the expected protein product is provided above as SEQ ID NO: 18. Both are shown and annotated in FIG. 10.

TABLE 5

| | | SEQ ID |
|---|---|---|
| Name | Sequence | NO: |
| OX40- | CCATGGAACATAACTGCTTTGGCAATACCTATCCGAAAG | 13 |
| 6His | ATGGCAAATGTTGCAATGATTGCCCGCCGGGTTATGGTAT | |
| optimized | GGAAAGCCGTTGCAGTCGTAGTCATGATACCAAATGTCA | |
| cDNA | TCAGTGTCCGAGTGGTTTTTATAATGAAGCCACCAATTAT | |
| with RS- | GAACCGTGTAAACCGTGCACCCAGTGTAATCAGCGCAGT | |
| 599 bp | GGCAGTGAACCGAAACGCCGCTGCACCCCGACCCAGGAT | |
| | ACCATTTGCAGCTGCAAACCGGGCACCGAACCGCGTGAT | |
| | GGTTATAAACGCGGTGTTGATTGTGCCCCGTGCCCGCCGG | |
| | GCCATTTTAGTCCGGGTGATGATCAGGCCTGTAAACCGTG | |
| | GACCAAACTGTATCTGATGAAACGTCGCACCATGCAGCC | |
| | GGCCAGCAAAAGTAGCGATGCAGTTTGCGAAGATCGTAG | |
| | CCTGCCGGCAACCCTGCCGTGGGAAACCCAGAGCCCGCT | |
| | GACCCGTCCGCCGACACCTCAGCCTACAATGGCTTGGCC | |
| | GCGCACCAGCCAGGGCCCTTTTACCCCGCCGACCGAACC | |
| | GCCGCGCGGTCCTCAGGGTAGTCATCATCATCATCACCAT | |
| | TAACTCGAG | |
| OX40- | MEHNCFGNTYPKDGKCCNDCPPGYGMESRCSRSHDTKCHQ | 18 |
| 6His | CPSGFYNEATNYEPCKPCTQCNQRSGSEPKRRCTPTQDTICS | |
| Protein- | CKPGTEPRDGYKRGVDCAPCPPGHFSPGDDQACKPWTKLY | |
| 196AAs- | LMKRRTMQPASKSSDAVCEDRSLPATLPWETQSPLTRPPTP | |
| 21.82 kDa | QPTMAWPRTSQGPFTPPTEPPRGPQGSHHHHHH | |

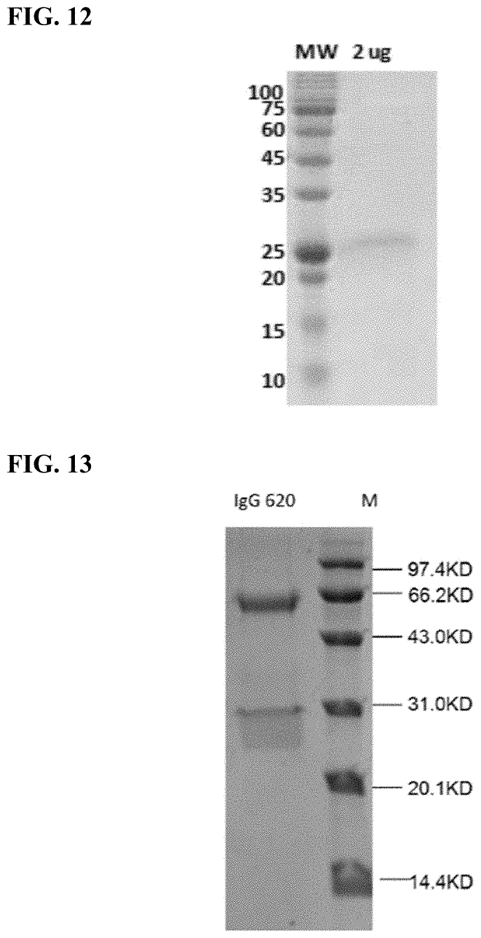

The full-length cDNA was then cloned into an expression vector (pcDNA 3.1). The complete vector and cDNA sequence is provided in FIG. 12 along with cloning sites for expression in the vector. The OX40 was then expressed and used to immunize mice and canines.

### Example 10: Immunization and Identification of Murine Anti-Canine OX40 IgG

In another exemplary method, Using Protocol 1 and 2, described below, four mice were immunized with the canine OX-40 protein 3 times [with 3 week intervals]. After the booster-1 and booster-2 immunization, the antisera were collected from each immunized mouse to monitor the immune response by titration ELISA, respectively. Good titers of antiserum against antigens were observed in all four mice, with mouse 4 having reached 1/102,400 after the booster-2 immunization. Then cell fusion was performed. Isolated spleen cells were fused with myeloma cells using PEG 1500. Then the fused cells were seeded into 96 wells plate directly by limited dilution. They were cultured in the presence of HAT medium. Three rounds of quantity control ELISA were performed to narrow down the original population (˜744 clones) to 5 stable positive clones (Table 6). The 5 positive clones identified were: 114, 412, 620, 560 and 340 and each was subcloned to search for additional stable hits. Of the 5 clones, only 3 showed stable positive results when subcloned (116, 412 and 620, Table 7). Following subcloning experiments, ascites antibody production for subclone 620 was conducted resulting in a total of 5 mg (1.25 mg/mL) antibodies obtained. Additional validation was performed on the antibody of subclone 620-91 purified from ascites. As shown in FIG. 13 and Table 8, the antibody showed high purity and a strong binding activity to its antigen was found using Western blot.

TABLE 6

| | Number of | Number of Positive |
|---|---|---|
| ELISA STEP | Clones Tested | Clones Identified |
| 1^{st} ELISA | 744 | 43 |
| 2^{nd} ELISA | 43 | 12 |
| 3^{rd} ELISA | 12 | 5 |

TABLE 7

| | Positive | Representative |
|---|---|---|
| Original | Subclones | positive subclones |
| Clone | (Y/N) | (purified from) |
| 114 | Y | |
| 412 | Y | |
| 620 | Y | 620-91 (ascites) |
| 560 | N | |
| 340 | N | |

TABLE 8

| Clones | A490 |
|---|---|
| 620-91 | 1.560 |
| normal serum (1:1,000) | 0.112 |
| positive serum (1:1,000) | 0.355 |
| PBS | 0.100 |

### Protocol I. Immunization

Four BALB/c mice (8 weeks old) were immunized intraperitoneally (i.p.). The emulsion was produced by complete mixing of pre-mixed antigen (17 μg/100 μL) with equal volume of adjuvant. Three immunizations were administered intraperitoneally at 3-week intervals. Before the first immunization and after the final immunization, serum samples were collected for subsequent serology analysis.

### Protocol 2. Titration ELISA

The wells of a 96 well plate were coated with canine OX-40 protein (0.5 μg/well) in a coating buffer and incubated overnight at 4° C. Following a wash step (3x with 200 μL PBST per well), the wells were then blocked with 300 μL PBSM for 1 hour at 37° C. The blocking buffer was removed and the plate washed 3 times with the washing buffer before 100 μL of antisera in blocking buffer was added per well. The plate was then incubated at 37° C. for 1 hour. Then the plate was washed again 3 times with a washing buffer before HRP-goat anti-mouse IgG (GE healthcare) (100 μL at 1:5,000 in blocking buffer) was added per well and incubated at 37° C. for 1 hour. Then the plate was washed again three times with the washing buffer. Tetramethylbenzidine (TMB) substrate (Sigma, USA) was diluted to 0.1 mmol/L citrate-phosphate buffer and combined with 1 μL/mL H₂O₂. 100 μL of the TMB substrate solution was added per well and incubated at room temperature for 15 minutes. Reaction was stopped using 100 μL of 2 mol/L H2504 solution. Plates were read at 490 nm.

### Protocol 3. Fusion Protocol

All mediums and PEG 1500 were pre-warmed to 37° C. FO myeloma cells were assessed, counted and left in RPMI1640+10% FCS in incubator throughout spleen cell recovery. For the spleen cell collection, a mouse was Euthanized via cervical dislocation before being placed in a beaker containing 200 mL of 80% Betadine, 20% 70%-ethanol. The spleen was removed using aseptic techniques and transferred to a Petri dish containing 5 mL RPMI1640+10% FCS. In a hood, the spleen was moved to a new Petri dish in 1 mL RPMI1640+10% FCS. The fat and connective tissue were trimmed, and the spleen cut into small pieces. Using two sterile watchmaker's forceps, a piece of spleen was secured while “milking” cells from the piece into the medium Collected cells were transferred to a 50 mL tube, through several (˜4) washes with 2-3 mL medium. The cell suspension was pipetted a few times, then let alone (˜1 min) until larger tissue pieces have fallen to the bottom of the tube. The upper cell suspension was collected, placed in new 50 mL tube and centrifuged at 900-1000 rpm for 5 min. The supernatant was removed, and spleen cells (pellet) were resuspended in 20 mL RPMI1640+10% FBS before they were counted (both undiluted and 1:10). Fusion was performed by first centrifuging a combination of mixed cells 900-1000 rpm for 5 minutes. Then the cells were washed with 25 mL RPMI1640 medium (no additives) and centrifuged again. The pellet was dislodged by finger-flicking to form a slurry of cells. Then 1.5 mL PEG was added per 3×10⁸mixed cells and the mixture incubated for 1 minute at 37° C. Then RPMI1640 was added very slowly up to a total volume of 20 mL. The reaction was centrifuged, and a calculated amount of HAT fusion medium was seeded onto 96 well plates directly.

### Example 11: Immunization and Identification of Additional Murine Anti-Canine OX40 IgG

In another exemplary method, five mice were immunized with the canine OX-40 protein 3 times (with 3-week intervals). After the third immunization, the antisera were collected from each immunized mouse to monitor the immune response by titration ELISA, respectively (see Protocol 1 in Example 10). Good titers of antiserum against antigens were observed in all five mice, with all titers having reached 1/121,500 after the 3rd immunization. Following titration, spleen cells were isolated and fused with myeloma cells using PEG 1500 (See Protocol 3 in Example 10) before the fused cells were seeded into 96 wells plate directly by limited dilution. They were cultured in the presence of HAT medium. In this example, all 960 single clones tested by ELISA were identified as positive. Another round of QC ELISA was performed by reducing the quantity of the target to 0.1 μg/well, but this also resulted in all positive clones. Accordingly, the 20 clones with the highest signal were selected and tested another two times using ELISA (see Table 9). All continued to test positive, so 10 clones having the highest signal from this set were then subcloned (see bolded values in Table 9) and stable clones were identified (Table 10). In addition, three clones from Table 9 (6B4, 5F5, and 9F3) were separately subcloned. In this set, subcloning 6B4 resulted in 2 stable clones while 5F5 and 9F3 each had 1 stable clone, resulting in a total of 4 stable clones identified (Table 11).

TABLE 9

| | The 3^{rd} | The 4^{th} | |
|---|---|---|---|
| Clones | QC ELISA | QC ELISA | |
| 1D8 | 3.510 | 3.595 | |
| 1F7 | 4.000 | 4.000 | Subcloning |
| 2C3 | 3.857 | 3.963 | |
| 2D9 | 3.510 | 3.810 | Subcloning |
| 3D11 | 3.069 | 3.333 | |
| 3E5 | 3.077 | 3.818 | Subcloning |
| 4C3 | 3.535 | 3.556 | |
| 4E8 | 3.540 | 3.818 | Subcloning |
| 5F5 | 3.342 | 3.833 | |
| 5E6 | 3.378 | 4.000 | Subcloning |
| 6B4 | 3.581 | 3.405 | |
| 6E7 | 3.120 | 3.517 | Subcloning |
| 7E5 | 3.217 | 3.347 | |
| 7E10 | 3.065 | 3.049 | Subcloning |
| 8E3 | 3.557 | 3.519 | Subcloning |
| 8E5 | 3.015 | 3.134 | |
| 9E4 | 3.209 | 3.362 | Subcloning |
| 9F3 | 3.357 | 3.415 | |
| 10C10 | 3.385 | 3.463 | |
| 10F6 | 4.000 | 4.000 | Subcloning |

TABLE 10

| | OD450 | |
|---|---|---|
| Clones | Test 1 | Test 2 |
| 1F7-1B4 | 3.115 | 3.336 |
| 1F7-1C4 | 3.428 | 3.513 |
| 1F7-1F2 | 3.836 | 4.000 |
| 3E5-3E11 | 3.345 | 3.306 |
| 5E6-5D5 | 3.268 | 3.199 |
| 6E7-6F1 | 3.310 | 3.348 |
| 7E10-7B8 | 3.198 | 3.167 |
| 7E10-7G4 | 3.224 | 3.354 |
| 10F6-10C4 | 3.771 | 3.828 |

TABLE 11

| | OD450 | |
|---|---|---|
| Clones | Test 1 | Test 2 |
| 6B4-1C8 | 3.658 | 3.701 |
| 6B4-1F1 | 3.701 | 3.689 |
| 5F5-2F10 | 3.415 | 3.662 |
| 9F3-3A6 | 3.530 | 3.465 |

### Example 12. Canine Immunization with OX40 and Isolation of Canine-Anti-Canine OX40 Antibodies

In another exemplary method, OX-40 was expressed as described above and used to immunize one canine and construct an immune library. A phage library screen was then used to identify specific binders for OX-40. In this system, the entire antibody library of the immunized canine was sequenced and cloned into a library of bacteria phages causing them to express the variable heavy (VH) and variable light (VL) chains as single chains on their surface (one VH/VL combination per phage). The phages were then biopanned to identify phages having an affinity for OX-40. (prior to panning the quality of OX-40 was tested by SDS-PAGE as illustrated in FIG. 13). As demonstrated in Table 10, after two rounds of screening obvious enriching effect was observed and clear difference between the sample screening and no-coating sample screening was found. Then a third round of biopanning was performed with a reduced concentration of the target OX-40 and increased washing strength the washing strength. As illustrated in Table 12, although the enriching effect was not obvious, the difference between the target screening group and no-coating control screening group clear.

TABLE 12

| Round | Conditions | Input | Output | Enriching factor |
|---|---|---|---|---|
| l^{st}-P | Target: OX-40 (75 μg/mL) | 1.50 × 10¹³ | 1.20 × 10⁸ | 1.25 × 10⁵ |
| | Blocking: 5% PBSM | | | |
| | Washing: 0.05% PBST 8 times and | | | |
| | PBS twice Elution: 0.25% Trypsin- | | | |
| | EDTA | | | |
| | Pre counter select: 5% PBSM | | | |
| l^{st}-N | Target protein: no coating Blocking: | 1.50 × 10¹³ | 1.00 × 10⁷ | 1.50 × 10⁶ |
| | 5% PBSM | | | |
| | Washing: 0.05% PBST 10 times and | | | |
| | PBS 4 times | | | |
| | Elution: 0.25% Trypsin-EDTA Pre | | | |
| | counter select: 5% PBSM | | | |
| 2^{nd}-P | Target: OX-40 (75 μg/mL) | 1.00 × 10¹³ | 4.00 × 10⁸ | 2.50 × 10⁴ |
| | Blocking: 5% PBSM | | | |
| | Washing: 0.05% PBST 10 times and | | | |
| | PBS 4 times | | | |
| | Elution: 0.25% Trypsin-EDTA Pre | | | |
| | counter select: 5% PBSM | | | |
| 2^{nd}-N | Target protein: no coating Blocking: | 1.00 × 10¹³ | 3.00 × 10⁵ | 3.33 × 10⁷ |
| | 5% PBSM | | | |
| | Washing: 0.05% PBST 10 times and | | | |
| | PBS 4 times | | | |
| | Elution: 0.25% Trypsin-EDTA Pre | | | |
| | counter select: 5% PBSM | | | |
| 3^{rd}-P | Target: OX-40 (37.5 μg/mL) | 5.20 × 10¹³ | 1.00 × 10⁷ | 5.20 × 10⁶ |
| | Blocking: 5% PBSM | | | |
| | Washing: 0.2% PBST 10 times and | | | |
| | PBS 5 times | | | |
| | Elution: 0.25% Trypsin-EDTA Pre | | | |
| | counter select: 5% PBSM | | | |
| 3^{rd}-N | Target protein: no coating Blocking: | 5.20 × 10¹³ | 6.00 × 10⁵ | 8.67 × 10⁷ |
| | 5% PBSM | | | |
| | Washing: 0.2% PBST 10 times and | | | |
| | PBS 5 times | | | |
| | Elution: 0.25% Trypsin-EDTA Pre | | | |
| | counter select: 5% PBSM | | | |

Enriching factor = input/output

Subsequently, 20 clones were isolated from the third round of elution outputs to validate the specificity of enrichment. As illustrated in Table 13, all 20 clones can bind to the target positively.

TABLE 13

| Clones | Coating: OX-40 | No coating |
|---|---|---|
| 1 (A1) | 1.6268 | 0.066 |
| 2 (A2) | 1.4727 | 0.0479 |
| 3 (A3) | 1.6178 | 0.0909 |
| 4 (A4) | 1.5219 | 0.0783 |
| 5 (A5) | 1.4701 | 0.0801 |
| 6 (A6) | 1.1337 | 0.0783 |
| 7 (A7) | 1.8345 | 0.0774 |
| 8 (A8) | 1.7009 | 0.0783 |
| 9 (A9) | 1.6983 | 0.0828 |
| 10 (A10) | 0.5043 | 0.0882 |
| 11 (C1) | 0.5542 | 0.0837 |
| 12 (C2) | 1.4023 | 0.0864 |
| 13 (C3) | 1.0924 | 0.1089 |
| 14 (C4) | 1.8289 | 0.0972 |
| 15 (C5) | 0.7409 | 0.0846 |
| 16 (C6) | 1.0136 | 0.0873 |
| 17 (C7) | 1.1517 | 0.0801 |
| 18 (C8) | 0.7639 | 0.0846 |
| 19 (C9) | 1.59 | 0.0792 |
| 20 (C10) | 0.7919 | 0.0855 |
| M13KO7 | 0.35 | 0.1098 |
| 1% M-PBS | 0.103 | 0.1071 |

DNA sequencing for all 20 positive clones was then performed to get the unique sequences. As illustrated in FIG. 14, 17 unique clones were sequenced successfully.

### Example 13. Sequencing of Candidate OX40 Antibody and Expression in HEK Cells

In another exemplary method, a full-length sequence of an illustrative OX40 antibody was generated. The full-length nucleic acid sequence of the variable heavy chain (VH) linked to the constant domain (CH123) and the variable light chain (VL) linked to the light constant domain (CK) is shown in Table 14, below.

TABLE 14

| Name | Sequence | SEQ ID NO: |
|---|---|---|
| VH-CH123 | ATGAGGGCCTGGATCTTCTTTCTCCTTTGCCTGGCCGGGAGGGCTCTGG | 14 |
| | CAGCCCCGCTAGCAGATGTGCTGCTCGTGGAAAGCGGCGGC | |
| | GATCTGGTGAAGCCCGGTGGCACACTGAGGCTGAGCTGTGTGGCCAGC | |
| | GGCTTCCCCTTCAGCAACTTCAACATGGGCTGGGTGAGGCAA | |
| | GCTCCCGGTAAAGGTTTACAGTGGGTGGCTTGGATTCATGGCAGCGGC | |
| | ATGACCACTCGTTACGCCGACGACGTGACCGGTCGTTTCACC | |
| | ATCTCTCGTGACAACGCCAAGGACACTTTATATTTAGAGATGGACTCTT | |
| | TAAGGCTGGAGGACACCGCCAAGTACTACTGCGCCAGAGAT | |
| | TTAGACGACGCCTATTTAGGCCCCAACTGGTTCAGCTACTGGGGCCAA | |
| | GGTACTTTAGTGATCGTGAGCAGCGCTAGCACCACGGCCCCC | |
| | TCGGTTTTCCCACTGGCCCCCAGCTGCGGGTCCACTTCCGGCTCCACGG | |
| | TGGCCCTGGCCTGCCTGGTGTCAGGCTACTTCCCCGAGCCT | |
| | GTAACTGTGTCCTGGAATTCCGGCTCCTTGACCAGCGGTGTGCACACCT | |
| | TCCCGTCCGTCCTGCAGTCCTCAGGGCTCTACTCCCTCAGC | |
| | AGCATGGTGACAGTGCCCTCCAGCAGGTGGCCCAGCGAGACCTTCACC | |
| | TGCAACGTGGCCCACCCGGCCAGCAAAACTAAAGTAGACAAG | |
| | CCAGTGCCCAAAAGAGAAAATGGAAGAGTTCCTCGCCCACCTGATTGT | |
| | CCCAAATGCCCAGCCCCTGAAATGCTGGGAGGGCCTTCGGTC | |
| | TTCATCTTTCCCCCGAAACCCAAGGACACCCTCTTGATTGCCCGAACAC | |
| | CTGAGGTCACATGTGTGGTGGTGGATCTGGACCCAGAAGAC | |
| | CCTGAGGTGCAGATCAGCTGGTTCGTGGACGGTAAGCAGATGCAAACA | |
| | GCCAAGACTCAGCCTCGTGAGGAGCAGTTCAATGGCACCTAC | |
| | CGTGTGGTCAGTGTCCTCCCCATTGGGCACCAGGACTGGCTCAAGGGG | |
| | AAGCAGTTCACGTGCAAAGTCAACAACAAAGCCCTCCCATCC | |
| | CCGATCGAGAGGACCATCTCCAAGGCCAGAGGGCAGGCCCATCAACC | |
| | CAGTGTGTATGTCCTGCCGCCATCCCGGGAGGAGTTGAGCAAG | |
| | AACACAGTCAGCTTGACATGCCTGATCAAAGACTTCTTCCCACCTGAC | |
| | ATTGATGTGGAGTGGCAGAGCAATGGACAGCAGGAGCCTGAG | |
| | AGCAAGTACCGCACGACCCCGCCCCAGCTGGACGAGGACGGGTCCTA | |
| | CTTCCTGTACAGCAAGCTCTCTGTGGACAAGAGCCGCTGGCAG | |
| | CGGGGAGACACCTTCATATGTGCGGTGATGCATGAAGCTCTACACAAC | |
| | CACTACACACAGGAATCCCTCTCCCATTCTCCGGGTAAATGA | |
| VL-CK | ATGAGGGCCTGGATCTTCTTTCTCCTTTGCCTGGCCGGGAGGGCTCTGG | 15 |
| | CAGCCCCGCTAGCAGACATCGTGATGACCCAAGCTCCTCCT | |
| | TCTTTATCTTTAAGCCCCGGTGAGCCCGCTAGCATCAGCTGCAAGGCC | |
| | AGCCAGTCTTTACTGCACAGCAACGGCAACACCTATTTATAC | |
| | TGGTTTCGTCAGAAGCCCGGACAGAGCCCCGAAGGTTTAATCTACAAG | |
| | GTGAGCGATCGTTTCACCGGCGTGAGCGACAGATTTAGCGGC | |
| | AGCGGTAGCGGCACCGATTTCACTTTAAGGATCTCTCGTGTGGAGGCC | |
| | GATGATGCCGGCGTGTACTACTGCGGCCAGAATTTACAGCTG | |
| | CCCTACAGCTTCAGCCAAGGTACCAAGCTGGAGATCAAGCGTACGGAT | |
| | GCCCAGCCAGCCGTCTATTTGTTCCAACCATCTCCAGACCAG | |
| | TTACACACAGGAAGTGCCTCTGTTGTGTGTTTGCTGAATAGCTTCTACC | |
| | CCAAAGACATCAATGTCAAGTGGAAAGTGGATGGTGTCATC | |
| | CAAGACACAGGCATCCAGGAAAGTGTCACAGAGCAGGACAAGGACAG | |
| | TACCTACAGCCTCAGCAGCACCCTGACGATGTCCAGTACTGAG | |
| | TACCTAAGTCATGAGTTGTACTCCTGTGAGATCACTCACAAGAGCCTG | |
| | CCCTCCACCCTCATCAAGAGCTTCCAAAGGAGCGAGTGTCAG | |
| | AGAGTGGACTAA | |

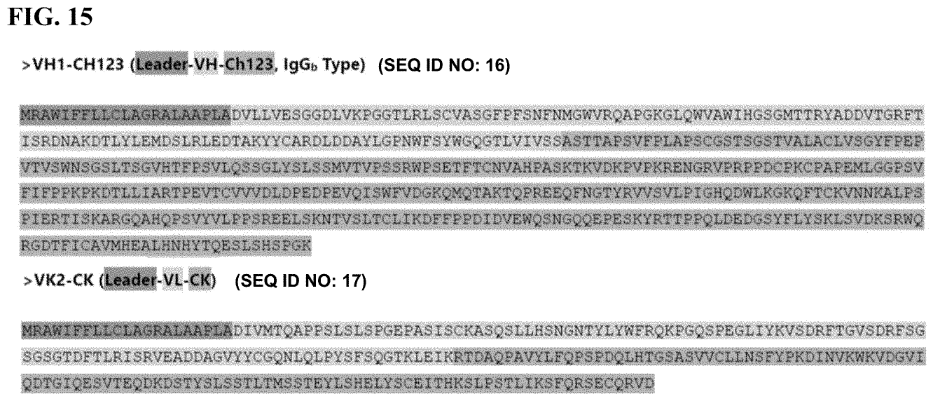

The corresponding amino acid sequence for the full-length antibody expressed by these constructs are provided in Table 15 below and in FIG. 15, which is also annotated with the variable and constant regions in each construct.

TABLE 15

| Name | Sequence | SEQ ID NO: |
|---|---|---|
| VH-CH123 | MRAWIFFLLCLAGRALAAPLADVLLVESGGDLVKPGGTLRLSCVASGFPF | 16 |
| | SNFNMGWVRQAPGKGLQWVAWIHGSGMTTRYADDVTGRFT | |
| | ISRDNAKDTLYLEMDSLRLEDTAKYYCARDLDDAYLGPNWFSYWGQGT | |
| | LVIVSSASTTAPSVFPLAPSCGSTSGSTVALACLVSGYFPEP | |
| | VTVSWNSGSLTSGVHTFPSVLQSSGLYSLSSMVTVPSSRWPSETFTCNVAH | |
| | PASKTKVDKPVPKRENGRVPRPPDCPKCPAPEMLGGPSV | |
| | FIFPPKPKDTLLIARTPEVTCVVVDLDPEDPEVQISWFVDGKQMQTAKTQP | |
| | REEQFNGTYRVVSVLPIGHQDWLKGKQFTCKVNNKALPS | |
| | PIERTISKARGQAHQPSVYVLPPSREELSKNTVSLTCLIKDFFPPDIDVEWQ | |
| | SNGQQEPESKYRTTPPQLDEDGSYFLYSKLSVDKSRWQ | |
| | RGDTFICAVMHEALHNHYTQESLSHSPGK | |
| VL-CK | MRAWIFFLLCLAGRALAAPLADIVMTQAPPSLSLSPGEPASISCKASQSLL | 17 |
| | HSNGNTYLYWFRQKPGQSPEGLIYKVSDRFTGVSDRFSG | |
| | SGSGTDFTLRISRVEADDAGVYYCGQNLQLPYSFSQGTKLEIKRTDAQPA | |
| | VYLFQPSPDQLHTGSASVVCLLNSFYPKDINVKWKVDGVI | |
| | QDTGIQESVTEQDKDSTYSLSSTLTMSSTEYLSHELYSCEITHKSLPSTLIKS | |
| | FQRSECQRVD | |

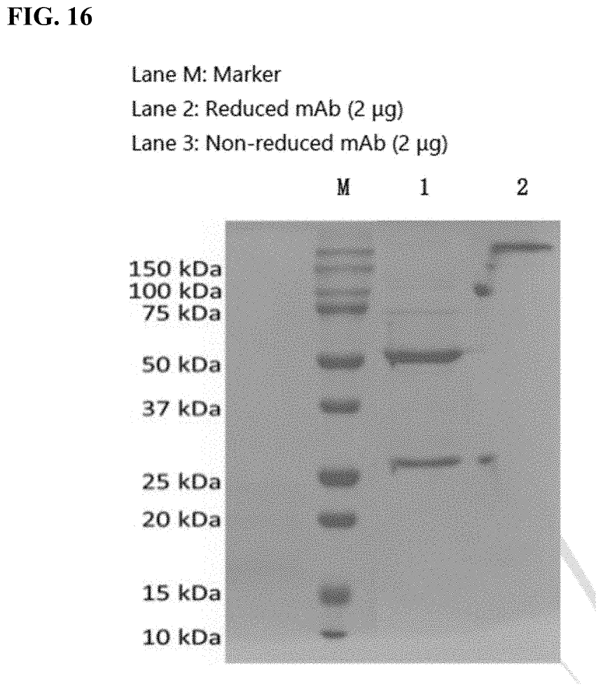

The illustrative antibody was expressed in HEK cells, isolated, and run on an SDS-page gel under non-reducing and reducing conditions. FIG. 16 shows the SDS-Page gel showing two bands in the reduced conditions and 1 band in the non-reduced condition. Table 16 below shows details about the sample that was run on SDS-Page. Protocols 1 and 2, below, were followed for the gene synthesis, subcloning and purification steps.

TABLE 16

| | Quantity | Concentration | Volume | | | |
|---|---|---|---|---|---|---|
| Sample | (mg) | (mg/mL) | (mL) | Vial | Buffer | Purity |
| Anti-OX40 | 1.06 | 0.53 | 2.0 | 4 | PBS, pH | >90% |
| IgGb | | | | (0.5 | 7.4 | |
| antibody | | | | mL/vial) | (5% | |
| (Clone F7) | | | | | glycerol) | |

### Protocol 1. Gene Synthesis and Subcloning in Expression Vector

The cDNAs of light chain and heavy chain of the antibody were chemically synthesized with optimization for mammalian expression. The cDNAs were cloned in expression vector.

### Protocol 2. 30 mL Scale-Up Purification Test

30 mL 293F cells with light chain and heavy chain co-transfected were cultured. The cells were collected with the best condition post transfection. Purification was done with affinity chromatography on protein A resin.

### Example 14: Combination Radiation Therapy and Immunotherapy

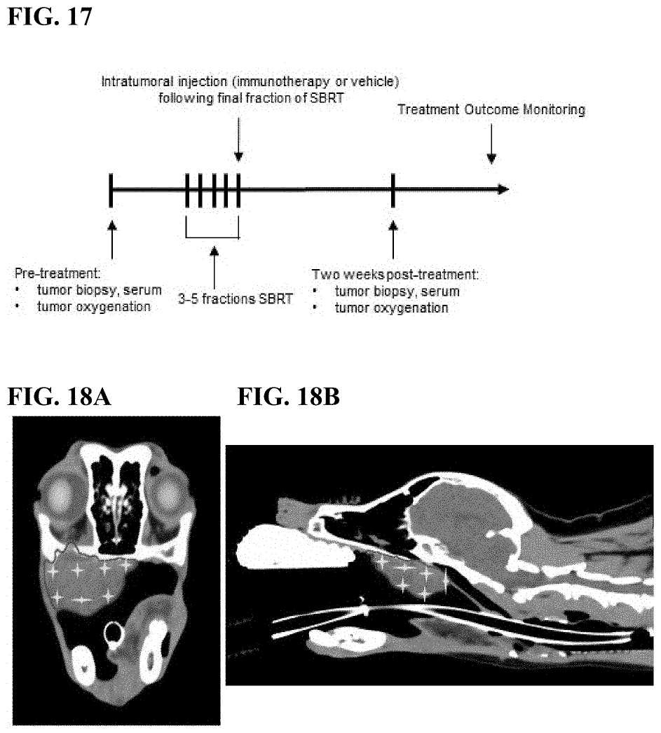

In another exemplary method, combination radiotherapy and immunotherapy were tested as cancer treatments in dogs. Twelve dogs were enrolled in the study, with 6 dogs randomized to each treatment group. The study design and injection CT-guided mapping approach are depicted in FIG. 17. In the SBRT only treatment group, there were 3 dogs with melanomas, 2 with carcinomas, 1 dog with a soft tissue sarcoma. In the SBRT+ Immunotherapy treatment group, there were 2 dogs with melanomas, 2 dogs with carcinomas, and 2 dogs with soft tissue sarcomas. Each animal received SBRT at a dose of 6-10 Gy per fraction for a total of 3-5 fractions each. The RT protocol decisions were based on standard clinical considerations, considering tumor type, size, and location.

The tumor locations, stage of disease, tumor volumes at the onset of treatment, and dose/volume of injections are described in Table 17. There was no difference in gross tumor volume (GTV) between dogs in the SBRT (mean 26.6 cm3, range 5.5-66 cm3) and SBRT+ immunotherapy (mean 47.3 cm3, range 2.3-112.7cm3) treatment groups (p=0.298). One dog in the SBRT group was treated with adjuvant chemotherapy (carboplatin), initiated 8 days post-RT; one dog in the SBRT+ immunotherapy received chemotherapy (carboplatin) 10 days prior to SBRT.

TABLE 17

| Signalment | | | | Stage of | |
|---|---|---|---|---|---|
| (MC: Male Castrated) | Tumor Type | Tumor | Injection Type | Disease at time | SBRT |
| (FS: Female Spayed) | and Location | Volume | and Volume | of treatment | protocol |
| 12 y MC | Melanoma | 5.5 cm³ | Vehicle control | Primary tumor | 10 Gy × 3 |
| Australian Terrier | (mandible) | | (PBS) | | |
| | | | (2 mL injections) | | |
| 13 y FS | Carcinoma | 66 cm³ | Vehicle control | Primary tumor, | 10 Gy × 3 |
| Mixed Breed | (salivary gland) | | (PBS) | Distant (pulmonary) | |
| | | | (3 mL injections) | metastasis | |
| 10 y MC | Melanoma | 51.9 cm³ | Vehicle control | Primary tumor | 10 Gy × 3 |
| Labrador Retriever | (maxilla) | | (PBS) | | |
| | | | (3 mL injections) | | |
| 3 y MC | Soft tissue | 8.6 cm³ | Vehicle control | Primary tumor | 6 Gy × 5 |
| German Shepherd | sarcoma | | (PBS) | | |
| | (mandible) | | (2 mL injections) | | |
| 9 y FS | Melanoma | 17.9 cm³ | Vehicle control | Primary tumor | 10 Gy × 3 |
| Maltese | (maxilla) | | (PBS) | | |
| | | | (3 mL injections) | | |
| 15 y MC | Carcinoma | 9.8 cm³ | Vehicle control | Primary tumor | 10 Gy × 3 |
| Mixed Breed | (maxilla) | | (PBS) | | |
| | | | (2 mL injections) | | |
| 13 y FS | Carcinoma | 30.1 cm³ | OX40/TLR agonists | Primary tumor, | 8 Gy × 5 |
| Miniature | (salivary gland) | | (200 mg OX40 in | Regional (nodal) | |
| Dachshund | | | 3 mL; 10 mg | metastasis | |
| | | | polyIC/10 mg | | |
| | | | pDNA in 3 mL) | | |
| 8 y MC | Soft tissue | 112.7 cm³ | OX40/TLR agonists | Primary tumor | 8 Gy × 5 |
| Mixed Breed | sarcoma | | (200 mg OX40 in | | |
| | (maxilla) | | 3 mL; 10 mg | | |
| | | | polyIC/10 mg | | |
| | | | pDNA in 3 mL) | | |
| 10 y MC | Melanoma | 2.3 cm³ | OX40/TLR agonists | Primary tumor | 10 Gy × 3 |
| Labrador Retriever | (maxilla) | | (100 mg OX40 in | | |
| | | | 1 mL; 5 mg | | |
| | | | polyIC/5 mg | | |
| | | | pDNA in 1 mL) | | |
| 10 y MC | Melanoma | 33.8 cm³ | OX40/TLR agonists | Primary tumor | 10 Gy × 3 |
| Mixed Breed | (maxilla) | | (200 mg OX40 in | | |
| | | | 3 mL; 10 mg | | |
| | | | polyIC/10 mg | | |
| | | | pDNA in 3 mL) | | |
| 10 y FS | Carcinoma | 35.5 cm³ | OX40/TLR agonists | Primary tumor | 10 Gy × 3 |
| Labrador Retriever | (mandible) | | (200 mg OX40 in | | |
| | | | 3 mL; 10 mg | | |
| | | | polyIC/10 mg | | |
| | | | pDNA in 3 mL) | | |
| 9 y FS | Soft tissue | 69.9 cm³ | OX40/TLR agonists | Primary tumor | 8 Gy × 5 |
| Miniature Poodle | sarcoma | | (200 mg OX40 in | | |
| | (axilla) | | 3 mL; 10 mg | | |
| | | | polyIC/10 mg | | |
| | | | pDNA in 3 mL) | | |

### Immune Infiltrates and Impact of SBRT and Immunotherapy

Regulatory T cell (FoxP3+) infiltrates in tumor tissues increased in tumors receiving SBRT only (mean density 0.44% pre-treatment vs 1.24% post-treatment, fold change: 2.24) whereas Treg density was reduced following treatment in animals treated with SBRT plus immunotherapy (mean density: 0.84% pre-treatment vs 0.423% post-treatment, fold change: 0.607) (FIG. 17). The difference in fold change in Treg density pre- to post-treatment between tumor treated with SBRT and SBRT plus immunotherapy was statistically significant (p=0.008). Tumor CD204+ macrophage infiltrate density increased in dogs receiving SBRT only (mean density: 7.57% pre-treatment vs 8.79% post-treatment, fold change: 1.51), whereas the density decreased in tumors from animals that received SBRT+ immunotherapy (mean density: 24.2%% pre-treatment vs 14.75% post-treatment, fold change: 0.50); the fold change in macrophage density was not significantly different between the treatment groups (p=0.151). FIGS. 18A-18B are representative three-dimensional injection maps which was prepared prior to injections for each dog in order to homogenously inject immunotherapy or vehicle (PBS). Red line outlines gross tumor volume; yellow stars indicate an injection site.

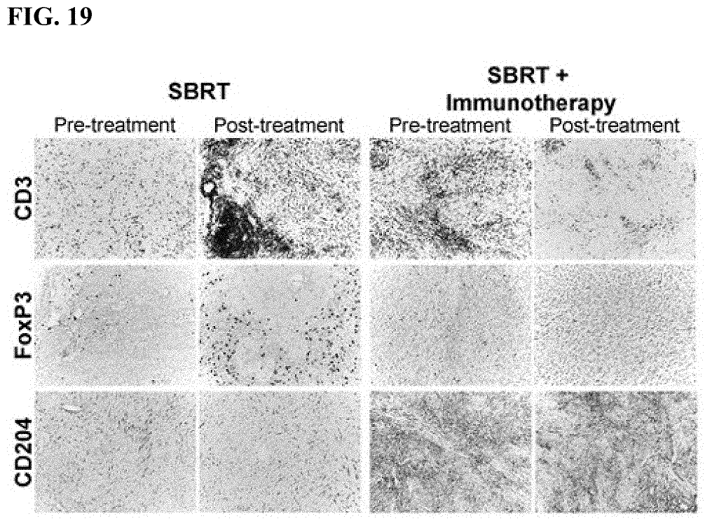

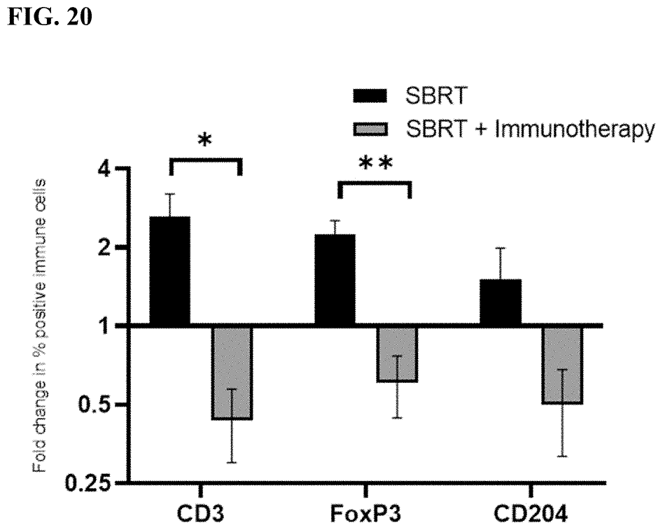

FIG. 19 provides representative immunohistochemistry (IHC) images of immune cell infiltrates, pre-treatment, and post-treatment, for tumors treated with SBRT or SBRT+ immunotherapy. FIG. 20 provides fold-change in percent positive immune cell density post-treatment relative to pre-treatment for tumors treated with SBRT or SBRT+ immunotherapy, with *p=0.025, **p=0.008 (SBRT: n=4; SBRT and immunotherapy: n=3). CD3+T cell density increased in the SBRT only group (mean density: 3.4% pre-treatment vs 10.1% post-treatment, fold change: 2.63), whereas paradoxically the CD3+ T cell density decreased in tumors that received SBRT+ immunotherapy (mean density 5.3%% pre-treatment vs 3.56% post-treatment, fold change: 0.44). The difference in fold change in CD3+ T cell density pre- to post-treatment between tumors treated with SBRT and SBRT plus immunotherapy was statistically significant (p=0.025). The Pax5+ B cell infiltrates did not change following SBRT or SBRT plus immunotherapy (fold change, p=0.955).

### Immune Gene Expression in Tumor Tissues and Impact of SBRT and Immunotherapy

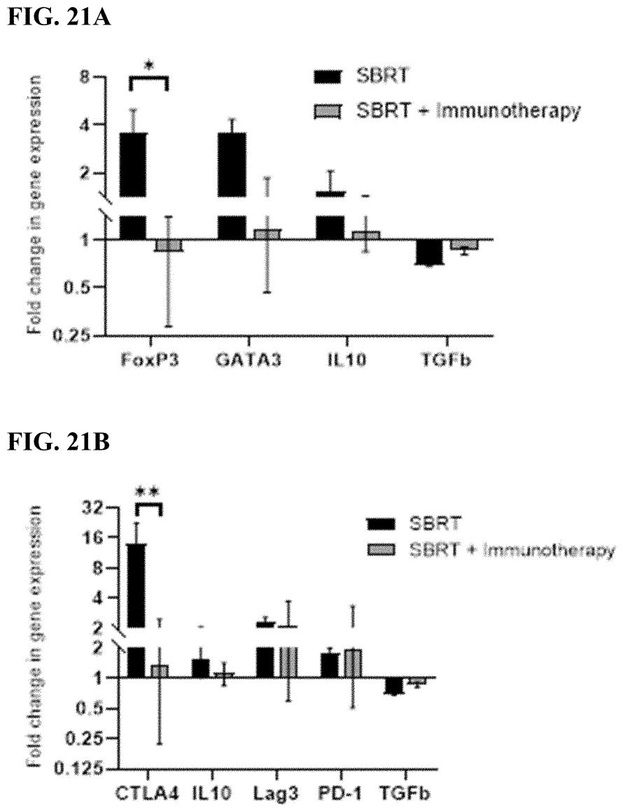

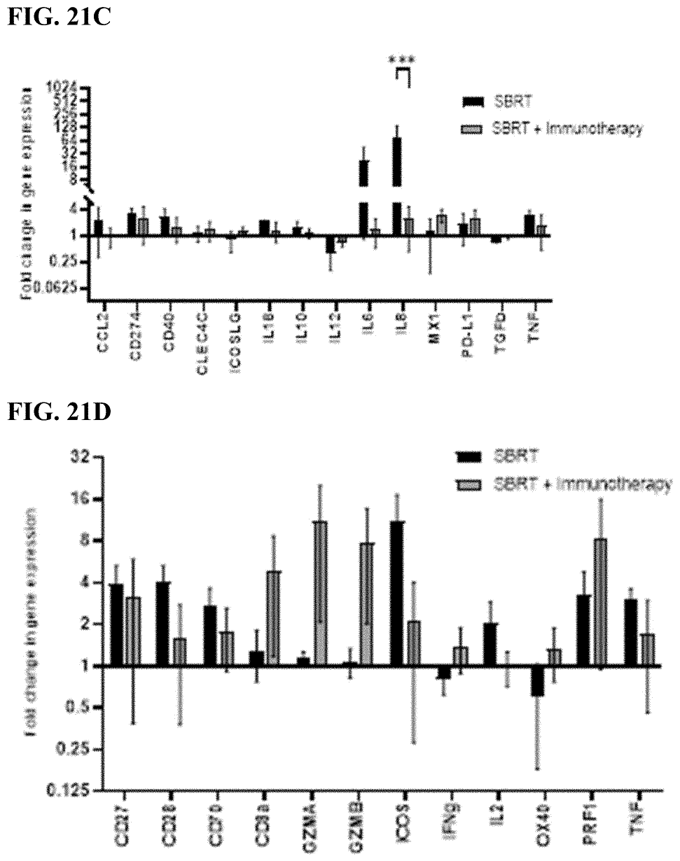

The impact of SBRT and immunotherapy on immune gene expression was evaluated using a custom Nanostring panel designed for quantification of expression of 45 genes related to cancer immune responses in dogs. Gene expression profiles were divided into patterns associated with Tregs, exhausted T cells, myeloid cells, and effector T cells. FIGS. 21A-D provide fold-changes in post-treatment relative to pre-treatment gene expression counts for tumors treated with SBRT or SBRT and immunotherapy via custom canine Nanostring immune panel (SBRT: n=2, SBRT+ immunotherapy: n=3). Gene expression profiles were divided into patterns associated with Regulatory T cell gene expression (FIG. 21A); exhausted T cell gene expression (FIG. 21B); myeloid cell gene expression (FIG. 21C); and effector T cell gene expression (FIG. 21D).

A significant difference in the fold-change of the Treg gene FoxP3 was identified in the Treg gene expression profile (FIG. 21A) (p=0.0378). Tumors treated with SBRT alone had increased expression of FoxP3 (mean: 52.0 pre-treatment vs. 161.9 post-treatment, fold change: 3.6) and tumors treated with SBRT+ immunotherapy had reduced expression of FoxP3 (mean: 246.9 pre-treatment vs. 103.2 post-treatment, fold change: 0.84). The fold-change in expression of CTLA4, a gene associated with negative regulation of T cell activation, was significantly different between the treatment groups in the exhausted T cell gene expression profile (FIG. 21B) (p=0.0054). Tumors treated with SBRT alone had significantly greater fold-change in CTLA4 expression (mean: 50.5 pre-treatment vs. 622.3 post-treatment, fold change: 14.0) while this effect was reduced in tumors treated with SBRT+ immunotherapy (mean: 338.0 pre-treatment vs. 273.1 post-treatment, fold change: 1.35). There were no significant findings in genes associated with activated myeloid cells (FIG. 21C); however, there was a significant decrease in the expression of IL-8 in tumors post-SBRT+ immunotherapy compared to SBRT alone (p=0.0001) (FIG. 21C). IL-8 is associated with immunosuppressive myeloid cells. Tumors treated with SBRT alone had increased expression of IL-8 (mean: 16950.0 pre-treatment vs. 122804.7 post-treatment, fold change: 72.6), while this effect was reduced in tumors treated with SBRT+ immunotherapy (mean: 723.8 pre-treatment vs. 1445.1 post-treatment, fold change: 2.5). No significant differences in effector T cell gene expression were revealed between treatment groups. Effector cell genes CD8a, GZMA, GZMB, IFNγ, OX40, and PRF-1 all had increased fold-change in expression following SBRT+immunotherapy compared to SBRT alone (FIG. 21D).

### Serum Cytokine Responses Following Treatment with SBRT or SBRT and Immunotherapy

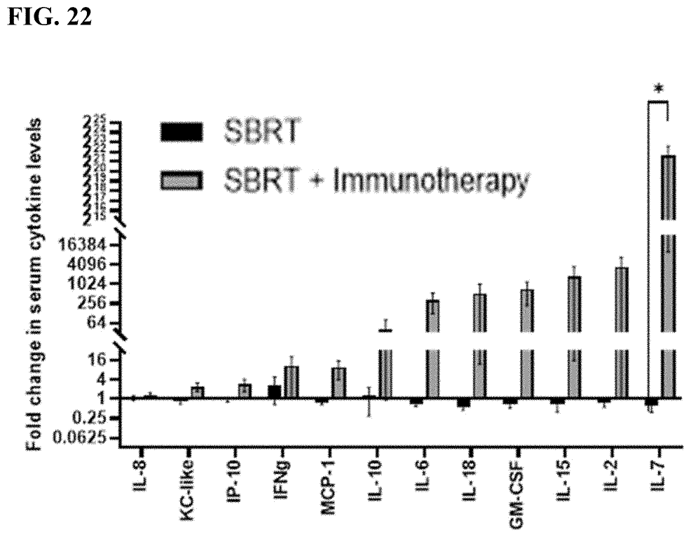

FIG. 22 illustrates data summarizing fold-change in serum cytokine levels post-treatment relative to pre-treatment for dogs treated with SBRT or SBRT+ immunotherapy (*p=0.035; SBRT n=6, SBRT and immunotherapy n=6). Of 13 cytokines assessed, only the fold change in serum IL-7 concentrations from pre- to post-treatment were significantly different between dogs treated with SBRT alone compared to SBRT plus immunotherapy. The fold change in serum IL-7 concentrations was significantly higher in animals treated with SBRT plus immunotherapy compared to SBRT only animals (SBRT fold change: 0.588 vs SBRT+immunotherapy fold change: 3.14×106, p=0.035).

### Impact of SBRT or SBRT and Immunotherapy on Tissue Vascularity and Oxygenation

The effects of SBRT and combination SBRT on immunotherapy regimens on tissue vascularity and oxygenation parameters were interrogated. To evaluate changes in the vascularity of the irradiated tissue microenvironment, tumor biopsy samples were analyzed pre- and post-treatment regardless of the percentage of viable tumor tissue in the section; however, one melanoma case in the SBRT and immunotherapy group was excluded from analysis.

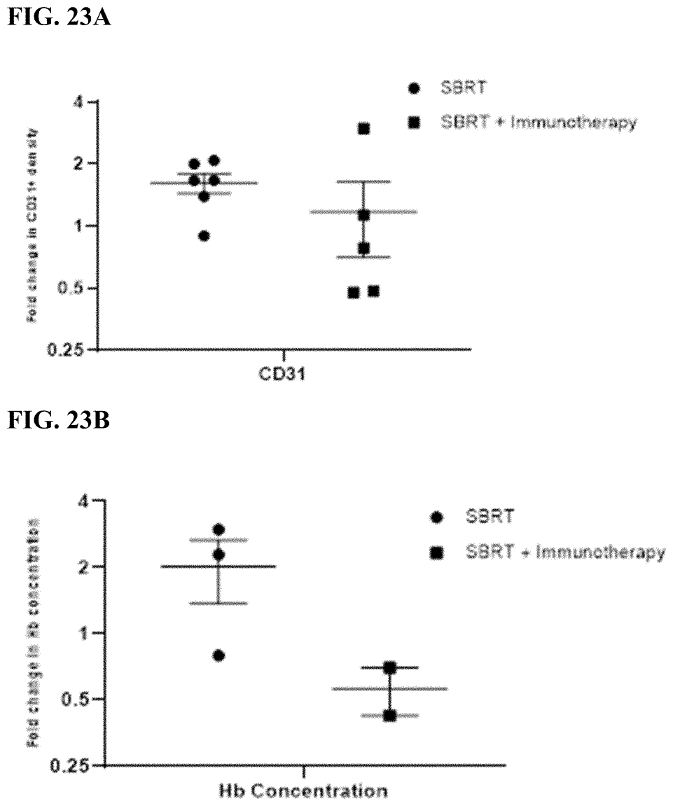

FIG. 23A illustrates data summarizing fold-changes in density of CD31+ endothelial cells post-treatment relative to pre-treatment samples for dogs treated with SBRT or SBRT+ immunotherapy (SBRT n=6, SBRT and immuno-therapy n=5). Tissue biopsies from sites treated with SBRT alone showed a mean 1.62 fold increase in CD31+ cell density. The fold increase in CD31+ cells in tissue biopsies treated with SBRT and immunotherapy was 1.17; however, this difference in fold change in CD31+ density was not statistically significant between treatment arms (p=0.515).

FIG. 23B illustrates data summarizing fold-changes in tumor hemoglobin saturation post-treatment relative to pre-treatment. (SBRT n=3, SBRT and immunotherapy n=2). There was no change in hemoglobin saturation in tumors treated with either SBRT (mean fold change: 1.01) or SBRT plus immunotherapy (mean fold change: 1.02) from pre-treatment to the two-week time point (p=0.344); however, there was a trend with respect to tumor hemoglobin concentration, as tumors treated with SBRT had a relative increase in Hb concentration at the two-week time point (mean fold change: 2.03) while tumors treated with SBRT and immunotherapy had a relative decrease in hemoglobin concentration at the two-week time point (mean fold change: 0.56). The difference in changes in hemoglobin concentration between the treatment groups did not reach statistical significance (p=0.15). Treatment responses to SBRT and SBRT and immunotherapy

The treatment response and outcome results of dogs evaluated in the study are provided as an overview of results found in Table 18. It should be noted that the study was not designed to assess the therapeutic efficacy of adding OX40/TLR agonist immunotherapy to SBRT, since only small numbers of animals were enrolled and only a single immunotherapy treatment was administered. Briefly, dogs treated with SBRT alone experienced a median PFS of 137 days (range: 75-260 days) and dogs treated with SBRT+ immunotherapy had a median PFS of 98 days (range: 19-433 days); dogs treated with SBRT alone had a median OST of 357 days (range: 147-477 days) and dogs treated with SBRT+ immunotherapy had a median OST of 208 days (range: 52-635 days). No statistically significant differences were found between treatment responses (p=0.567), PFST (p=0.575), and OST (p=0.943) for dogs treated with SBRT compared to SBRT+ immunotherapy.

TABLE 18

| Signalment | | | | | | |
|---|---|---|---|---|---|---|
| (MC: male | | | Progression | | | |
| castrated; | Tumor | | Free | | Overall | |
| FS: female | Type and | Treatment | Survival | Pattern of | Survival | |
| spayed) | Location | Response | (days) | Failure | Time (days) | Cause of Death |
| 12y MC | Melanoma | Progressive | 75 | Local, | 452 | Local, Regional, |
| Australian | (mandible) | disease | | Regional, | | Distant |
| Terrier | | | | Distant | | |
| 13y FS | Carcinoma | Stable | 115 | Distant | 477 | Local, Regional, |
| Mixed Breed | (salivary | disease | | | | Distant |
| | gland) | | | | | |
| l0y MC | Melanoma | Complete | 147 | Local | 147 | Local |
| Labrador | (maxilla) | response | | | | |
| Retriever | | | | | | |
| 3y MC | Soft tissue | Partial | 127 | Local | 262 | Local |
| German | sarcoma | response | | | | |
| Shepherd | (mandible) | | | | | |
| 9y FS | Melanoma | Complete | 260 | Local | 371 | Alive at the time |
| Maltese | (maxilla) | response | | | (alive at the | of analysis |
| | | | | | time of | |
| | | | | | analysis) | |
| 15y MC | Carcinoma | Complete | 154 | Local | 162 | Local |
| Mixed Breed | (maxilla) | response | | | | |
| 13y FS | Carcinoma | Progressive | 19 | Regional | 52 | Regional (out- |
| Miniature | (salivary | disease | | (out-of- | | of-field) |
| Dachshund | gland) | | | field) | | |
| 8y MC Mixed | Soft tissue | Stable | 114 | Distant | 224 | Distant |
| Breed | sarcoma | disease | | | | |
| | (maxilla) | | | | | |
| l0y MC | Melanoma | Partial | 260 | Local, | 635 | Local, Regional, |
| Labrador | (maxilla) | response | | regional | | Distant |
| Retriever | | | | (nodal), | | |
| | | | | distant | | |
| l0y MC | Melanoma | Partial | 82 | No evidence | 82 | Other |
| Mixed Breed | (maxilla) | response | | of | | (Acute collapse) |
| | | | | progression | | |
| | | | | at time of | | |
| | | | | death | | |
| 10y FS | Carcinoma | Progressive | 79 | Regional | 192 | Other |
| Labrador | (mandible) | disease | | (out-of- | | (Acute kidney |
| Retriever | | | | field) | | injury) |
| 9y FS | Soft tissue | Stable | 433 | No evidence | 433 | Alive at the time |
| Miniature | sarcoma | disease | (no | of | | of analysis |
| Poodle | (axilla) | | progression) | progression | | |
| | | | | at time of | | |
| | | | | analysis | | |

There were no statistical differences in the toxicity profiles across dogs in either treatment group (acute: p>0.99, delayed/late: p>0.99, incidence of severe toxicity: p>0.99, incidence of any toxicity: p>0.99)

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. An isolated anti-canine OX40 activating antibody wherein the antibody induces OX40 activities.

2. The isolated anti-canine OX40 activating antibody according to claim 1, wherein the antibody comprises a V_{H} comprising 85% or more sequence identity to a V_{H} of monoclonal antibody clone 7E10F3 or monoclonal antibody clone CAF7 and a V_{L} comprising 85% or more sequence identity to a V_{L} of monoclonal antibody clone 7E10F3 or monoclonal antibody clone CAF7 wherein the isolated antibody does not inhibit OX40 activity.

3. The isolated anti-canine OX40 activating antibody according to claim 1, wherein the antibody is monoclonal antibody clone 7E10F3 or monoclonal antibody clone CAF7.

4. The isolated anti-canine OX40 activating antibody according to claim 1, wherein the antibody specifically binds to an epitope on a polypeptide having an amino acid sequence represented by SEQ ID NO: 1 or SEQ ID NO: 19.

5. (canceled)

6. The isolated anti-canine OX40 activating antibody according to claim 1, wherein the antibody is a full-length antibody or an antigen binding fragment thereof.

7. The isolated anti-canine OX40 activating antibody according to claim 1, wherein the antibody is a monoclonal antibody or a single-chain antibody (scFv).

8. (canceled)

9. A polynucleotide encoding an isolated anti-canine OX40 activating antibody according to claim 2 or a fragment thereof.

10. The polynucleotide according to claim 9, further comprising a vector for expressing the encoded activating anti-canine antibody.

11. A host cell comprising the polynucleotide according claim 9.

12-13 (canceled)

14. A pharmaceutical composition comprising the isolated anti-canine OX40 activating antibody according to claim 1, and a pharmaceutically acceptable carrier.

15. The pharmaceutical composition according to claim 14, further comprising a non-specific innate immune response stimulator.

16. The pharmaceutical composition according to claim 15, wherein the non-specific innate immune response stimulator comprises at least one of a cationic liposome, a TLR ligand, and a cellular adhesion agent.

17. The pharmaceutical composition according to claim 14, further comprising at least one of an anti-microbial agent, a chemotherapeutic agent, and an anti-PD-1 antibody.

18. (canceled)

19. A method for inducing innate immunity in a canine, comprising administering to a canine in need thereof the pharmaceutical composition according to claim 14.

20. A method of treating cancer in a canine, comprising administering the pharmaceutical composition according to claim 14 to the canine and treating cancer in the canine.

21. The method according to claim 20, wherein the cancer comprises a solid tumor.

22. The method according to claim 20, further comprising treating the canine with radiation therapy at least one of before, during and after administering the pharmaceutical composition.

23. The method according to claim 20, further comprising administering one or more agent for inducing a non-specific immune response in the canine.

24. A method for treating immunosuppression in a canine comprising administering the pharmaceutical composition according claim 14 to the canine and treating immunosuppression in the canine.

25. The method according to claim 24, wherein the immunosuppression is caused by acute or chronic microbial or mixed microbial infection.
